(19) United States
(12) Patent Application Publication
(10) Pub. No.: US 2007/0061898 A1
(43) Pub. Date: Mar. 15, 2007

(54) Transgenic mouse expressing a polynucleotide encoding human tissue factor

(76) Inventors: Jing Yang, Ambler, PA (US); Robert Jordan, Thornton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/445,777

(22) Filed: Jun. 2, 2006

Related U.S. Application Data
(60) Provisional application No. 60/688,607, filed on Jun. 8, 2005.

(51) Int. Cl. A01K 67/027

(52) U.S. Cl. 800/3; 800/18

(57) ABSTRACT

Transgenic mice expressing human tissue factor which are useful for evaluating pharmacologic agents which interact with human tissue factor and for investigating diseases associated with human tissue factor.

This present application claims the benefit of priority to U.S. Provisional Application Ser. No. 60/688,607 filed Jun. 8, 2005. The application submitted herewith contains a Sequence Listing on computer readable disk which material is hereby incorporated by reference.

### BACKGROUND OF THE INVENTION

1. Field of the Invention

The present invention relates to field of animal models useful for drug development. More specifically, the invention relates to transgenic mice expressing human tissue factor which are useful for evaluating pharmacologic agents which interact with human tissue factor and for investigating diseases associated with human tissue factor.

2. Background

The coagulation of blood involves a cascading series of reactions leading to the formation of fibrin. The coagulation cascade consists of two overlapping pathways, both of which are required for hemostasis. The intrinsic pathway comprises protein factors present in circulating blood, while the extrinsic pathway requires tissue factor (TF), which is expressed on the cell surface of a variety of tissues in response to vascular injury (Davie et al., 1991, Biochemistry 30:10363). When exposed to blood, TF sets in motion a rapid cascade of activation steps that result in the formation of an insoluble fibrin clot (See FIG. 1).

TF has been investigated as a target for anticoagulant therapy. TF (also known as thromboplastin, CD142 and coagulation factor III) is a single chain, 263 amino acid membrane glycoprotein that functions as a receptor for factor VII and VIIa and thereby initiates the extrinsic pathway of the coagulation cascade in response to vascular injury. TF is an integral membrane protein normally present on the cell surface of non-vascular cell types. Healthy endothelial cells lining normal blood vessels do not produce TF, however, TF is always present in the adventitia of blood vessels.

TF serves as both a cofactor for factor VIIa, forming a proteolytically active TF:VIIa complex on cell surfaces, and as a VIIa receptor, inducing downstream intracellular changes (Bazan, J F, Proc. Natl. Acad. Sci USA (1990) 87:6934-8; Reviewed by Konigsberg, et al. Thromb. Haemost. (2001) 86:757-71). In addition to its role in the maintenance of hemostasis by initiation of blood clotting, TF has been implicated in pathogenic conditions. Specifically, the synthesis and cell surface expression of TF has been implicated in vascular disease (Wilcox et al, 1989, Proc. Natl. Acad. Sci. 86:2839) and gram-negative septic shock (Warr et al., 1990, Blood 75:1481). Furthermore, in a number of pathological states involving an acute inflammatory response and progression to a thrombotic state, such as sepsis, increased TF expression on the vascular endothelium results from the release of inflammatory mediators, such as TNF and/or IL-1.

The Role of TF in Cancer

Tissue factor is also overexpressed on a variety of malignant tumors and isolated human tumor cell lines, suggesting a role in tumor growth and survival. TF is not produced by healthy endothelial cells lining normal blood vessels but is expressed on these cells in tumor vessels. It appears to play a role in both vasculogenesis in the developing animal and angiogenesis in normal and malignant adult tissues. Inhibition or targeting of TF may therefore be a useful anti-tumor strategy that could affect the survival of TF overexpressing tumor cells directly by inhibiting TF mediated cellular signaling or other activities. In addition, this approach may prevent tumor growth indirectly via an antiangiogenic mechanism by inhibiting the growth or function of TF expressing intra-tumoral endothelial cells.

WO94/05328 discloses the use of anti-TF antibodies to inhibit the onset and progression of metastasis by abolishing the prolonged adherence of metastazing cells in the microvasculature thereby inhibiting metastasis, but does not disclose any effect on the growth of established tumor cells.

The Role of TF in Transplantation

Despite the central role of TF in blood coagulation, the mechanisms underlying the regulation of TF pro-coagulant activity in vivo are still being explored as are non-coagulant activities related to receptor signaling (Morrissey, J. H. Thromb Haemost 2001; 86:66-74 and Key, N. S., Bach, R. R. Thromb Haemost 2001; 85:375-6).

Unperturbed cells in culture have weak coagulant activity, however, cells or tissues that have been disrupted or stimulated with, e.g. growth factors or endotoxin leading to increased intracellular calcium ion (Ca++), display fully expressed and active TF. Perturbation of the phospholipid species between the inner and outer cell membrane leaflets, especially phosphatidyl serine, was implicated as a possible trigger of this de-encryption of a macromolecular substrate binding site on TF which defines TF activation (Bach, R. R, Moldow, C. F. Blood 1997; 89 (9): 3270-3276).

A number of reports implicate the role of active TF in the pathogenesis of transplant failure. U.S. Pat. No. 6,387,366 notes that bone marrow stem cell (BMSC) transplantation causes blood clotting or hemorrhage due to the expression of TF on the infused cells and suggests several methods to reduce the biological activity of TF or FVII in infusions employing BMSC transplantation, gene therapies employing BMSC, and other types of cell transplantation. These methods include treating the preparation or the patient with TF antagonists.

TF Antagonists

Various anti-TF antibodies are known. For example, Carson et al, Blood 70:490-493 (1987) discloses a monoclonal antibody prepared from hybridomas produced by immunizing mice with human TF purified by affinity chromatography on immobilized factor VII. Ruf et al, (1991, Thrombosis and Haemostasis 66:529) characterized the anticoagulant potential of murine monoclonal antibodies against human TF. The inhibition of TF function by most of the monoclonal antibodies that were assessed was dependent upon blocking the formation or causing the dissociation of the TF/VIIa complex that is rapidly formed when TF contacts plasma. Such antibodies were thus relatively slow inhibitors of TF in plasma as factor VII/VIIa remains active. One monoclonal antibody, TF8-5G9, was capable of inhibiting the TF/VIIa complex by blocking the F.X binding site without dissociating the complex, thus providing an immediate anticoagulant effect in plasma which is not absolute as F.VII is still available. This antibody is disclosed in U.S. Pat. Nos. 6,001,978, 5,223,427, and 5,110,730. Ruf et al. suggest that mechanisms that inactivate the TF/VIIa complex, rather than prevent its formation, may provide strategies for interruption of coagulation in vivo. In contrast to other antibodies that inhibit factor VII binding to TF, TF8-5G9 shows only subtle and indirect effects on factor VII or factor VIIa binding to the receptor. TF8-5G9 binds to defined residues of the extracellular domain of TF that are also involved in F.X binding with a nanomolar-binding constant. Thus, TF8-5G9 is able to effectively block the subsequent critical step in the coagulation cascade, the formation of the TF:VIIa:X ternary initiation complex (Huang et al, J. Mol. Biol. 275:873-894 1998).

Anti-TF monoclonal antibodies have been shown to inhibit TF activity in various species (Morrissey et al, Throm. Res. 52:247-260 1988) and neutralizing anti-TF antibodies have been shown to prevent death in a baboon model of sepsis (Taylor et al, Circ. Shock, 33:127 (1991)), and attenuate endotoxin induced DIC in rabbits (Warr et al, Blood 75:1481 (1990))

WO 96/40921 discloses CDR-grafted anti-TF antibodies derived from the TF8-5G9 antibody. Other humanized or human anti-TF antibodies are disclosed in Presta et al, Thromb Haemost 85:379-389 (2001), EP1069185, U.S. Pat. No. 6,555,319, WO 01/70984 and WO03/029295.

An antibody that specifically recognizes TF and inhibits coagulation may provide a useful therapy for diseases where thrombogenesis is abnormal. However, to evaluate the potential efficacy of an anti-TF antibody in vivo, the antibody must cross react with TF from the animal or a surrogate must be identified that acts in a similar manner to the anti-human TF antibody. In vitro experiments have demonstrated that the anti-human TF antibody, 5G9, does not bind to murine TF. This observation is consistent with the structural data and with the differences in the mouse and human proteins in the region that constitutes the 5G9 epitope. Indeed, there are eight residues within the epitope that are different between murine and human TF. Efforts to generate an anti-murine TF antibody that acts in a manner similar to 5G9 by immunization of rats or other animals have not heretofore been successful.

Thus, there exists a need for a non-human animal model that expresses human tissue factor and methods of identifying therapeutic agents that interact with human tissue factor and are thus useful in treating diseases associated with human tissue factor. Transgenic mice, expressing <1% of human TF activity, have been reported using a human tissue factor minigene vector, human TF minigene promoter and human coding sequence, Parry et al JCI 101(3):560-569, 1998.

### SUMMARY OF THE INVENTION

The invention provides a transgenic non-human mammal comprising nucleated cells containing a transgene encoding a human tissue factor polypeptide. The polynucleotide encoding human tissue factor polypeptide is operably linked to the endogenous expression control sequence that includes the tissue factor promoter of the non-human mammal. In particular, the transgenic non-human mammal can be a mouse where the transgene encoding a human tissue factor polypeptide is operably linked to the murine tissue factor promoter. The invention also provides methods of using such a transgenic non-human mammal expressing human tissue factor to identify a therapeutic agent for use in treating a tissue factor related disease by administering a compound to such a transgenic non-human mammal expressing human tissue factor and screening the transgenic non-human mammal for an improved response associated with a tissue factor related phenotype of the transgenic non-human mammal, thereby identifying a therapeutic agent for use in treating the tissue factor related disease.

In one aspect, the transgenic non-human mammal containing the transgene is useful as a surrogate in preclinical testing in non-human mammal hosts of therapeutic candidates, such as antibodies, where the therapeutic candidate for use in humans does not bind with the homolog non-human tissue factor target protein in a manner that would provide meaningful information about treatment efficacy or safety. The invention provides a model for use in pre-clinical testing of therapeutic biologic candidates in animal models.

In one embodiment, the transgene comprises a polynucleotide encoding human tissue factor.(SEQ. ID. NO. 2). The polynucleotide is operably linked to an expression control sequence that includes the endogenous promoter, capable of expressing a DNA sequence that is substantially identical to that of SEQ. ID NO. 2. In a particular embodiment, the non-human animal is a mouse, the transgene is the polynucleotide encoding human tissue factor(SEQ. ID. NO. 2) and it is operably linked to the murine tissue factor promoter. Thus, in this embodiment, the invention relates to a mouse TF gene construct encoding human TF, instead of mouse TF, at the initiation codon. The human TF sequence is “embedded” at the mouse TF locus by homologous recombination, thus allowing expressing of huTF by the endogenous mouse TF promoter. In this manner the human TF is expressed and regulated just like the endogenous mouse TF, such as in the brain, heart and uterus and on endothelial cells and monocytes upon LPS stimulation.

In an additional embodiment, the human tissue factor is encoded by a polynucleotide that hybridizes under high stringency conditions to the coding sequence of human tissue factor. Desirably, the expression is controlled such that the human tissue factor is expressed in perivascular cells and epithelial cells. Most desirably, the human tissue factor expression is inducible in circulating monocytes and endothelial cells in a variety of pathological conditions, including gram-negative sepsis.

In another embodiment, the mouse can be a CD-1® Nude mouse, a CD-1 mouse, a NU/NU mouse, a BALB/C Nude mouse, a BALB/C mouse, a NIH-III mouse, a SCID™ mouse, an outbred SCID™ mouse, a SCID Beige mouse, a C3H mouse, a C57BL/6 mouse, a DBA/2 mouse, a FVB mouse, a CB17 mouse, a 129 mouse, a SJL mouse, a B6C3F1 mouse, a BDF1 mouse, a CDF1 mouse, a CB6F1 mouse, a CF-1 mouse, a Swiss Webster mouse, a SKH1 mouse, a PGP mouse, or a B6SJL mouse. In a preferred embodiment, the transgenic mouse whose genome contains a polynucleotide encoding human tissue factor further contains a homozygous deletion or disruption of the murine tissue factor gene. Thus, the mouse expresses the human tissue factor but not the murine tissue factor protein.

Another aspect of the invention features a cell line derived from a transgenic mouse of the invention.

In yet another aspect, the invention features a method for producing a transgenic mouse by providing an exogenous expression vector that contains a nucleotide sequence of the mouse tissue factor promoter operably linked to a nucleotide sequence encoding the human tissue factor. In one embodiment, the expression vector is introduced into a fertilized mouse oocyte, which is then allowed to develop to term. The desired mouse is characterized by expression of human tissue factor. In another aspect, the invention features a method for screening or characterization of one or more compounds or pharmaceutical compositions for their ability to inhibit human tissue factor procoagulant activity using a transgenic mouse that produces in its perivascular cells, epithelial cells, endothelial cells or monocytes recombinant human tissue factor.

### BRIEF DESCRIPTION OF THE DRAWINGS

FIG. 1. Generation of huTF knock-in mice. The knock-in contruct and the homologous recombination strategy is shown. The genomic fragment containing exon 1 and 2 of mouse TF was deleted and replaced by an expression cassette IRES/huTF/MCl Neo. The open reading frame of huTF was inserted in-frame at the initiation codon of mouse tissue factor gene. The neomycin resistance gene (Neo) and the HSV-thymidine kinase gene were included for positive and negative selection. The Neo gene is flanked by loxP site and is excised upon germline transmition.

FIG. 2. Normal expression pattern of huTF in (h/h) mice

A. HuTF expression as measured by real time RT-PCR. Equal amounts of RNA were pooled from the tissues of three mice for analysis. Expression is shown as percent of brain control, where brain=100%. Top panel: Expression of mouse TF in (h/h) (KI) and (m/m) (WT) mice. Bottom panel: Expression of human TF in (h/h) (KI) and (m/m) (WT) mice.

B. Western blot of huTF protein in (h/h) and (m/m) mice. Top panel: goat anti-human TF antibody; Bottom panel: rabbit anti-mouse TF antibody. K=extract from (h/h) mice; W=extract from (m/m) mice.

FIG. 3. Functional human TF is expressed from the (h/h) mice. The prothrombin time of brain extract from (h/h) mice and (m/m) mice (A) or (h/h) mice and human sample (B) are shown. Pro-coagulant activity was measured with a one stage clotting assay.

FIG. 4. The procoagulant activity of human tissue factor from (h/h) mice is inhibited by anti-human tissue factor antibody CNT0859. Dose-dependent inhibition of brain extract by anti-human tissue factor monoclonal antibody CNT0859 (A, C) or a anti-mouse tissue factor antibody PHD167 (B) are shown. Brain extract from (h/h) mice vs (m/m) mice (A, C) or (h/h) mice vs human sample (C) are indicated. Brain extracts were diluted to their EC₅₀ in HBSS and 15 mM CaCl₂. Each extract was then incubated with increasing concentrations of CNTO 859 or CNTO 126. Pro-coagulant activity was measured with a one stage prothrombin assay.

FIG. 5. Normal hemostasis in the (h/h) mice. The tail bleeding time in (h/h) mice and the (m/m) mice are given.

### DETAILED DESCRIPTION OF THE INVENTION

The present invention provides a transgenic non-human mammal, in particular a transgenic mouse, comprising nucleated cells containing a transgene encoding a human tissue factor polypeptide which is operably linked to the endogenous expression control sequence that includes the tissue factor promoter of the non-human mammal. In particular, the transgenic non-human mammal can be a mouse wherein the transgene encoding a human tissue factor polypeptide is operably linked to the murine tissue factor promoter. The transgenic non-human mammal of the invention is useful as a model for tissue factor related disease. A transgenic non-human mammal expressing the encoded tissue factor polypeptide, or cells derived therefrom, can also be advantageously used in methods to identify therapeutic agents useful for treating various tissue factor related diseases, for example, vascular disease, sepsis and cancer.

The present invention provides a mutant non-human mammal comprising nucleated cells containing a transgene encoding a tissue factor polypeptide. As disclosed herein, a “mutant” refers to a genetic change, for example, a mutant form of a nucleic acid or encoded polypeptide means that the nucleic acid contains a genetic modification relative to a parent nucleic acid such as the wild type form of the nucleic acid. Similary, a “mutant,” when used in reference to an animal refers to an animal that has been genetically modified. The genetic modification can be the insertion of a gene, thereby generating a “transgenic” animal. As used herein, a “transgene,” when used in reference to a transgenic animal, refers to a gene that is inserted into the germ line of an animal in a manner that ensures its function, replication, and transmission as a normal gene, also referred to as a “knock-in” animal. A mutant animal of the invention can be any non-human mammal such as a mouse. A mutant animal can also be, for example, other non-human mammals such as rat, rabbit, goat, pig, guinea pig, sheep, cow, non-human primate or any non-human mammal. It is understood that mutant animals expressing a human tissue factor transgene, in addition to the tissue factor mutant mouse disclosed herein, can be used in methods of the invention. In one embodiment of the invention mutant mammal, a human tissue factor gene was introduced into a mouse to generate a transgenic mouse expressing human tissue factor. (see Example 1).

A transgenic non-human mammal of the invention contains a transgene encoding a human tissue factor polypeptide or variant thereof. The human tissue factor polypeptide can be encoded by a human gene, or a splice variant thereof. For example, a nucleic acid encoding a human tissue factor polypeptide can be a nucleic acid sequence of SEQ ID NO. 2.

A human tissue factor polypeptide can be, for example, the human amino acid sequence referenced as SEQ ID NO.1. As used herein, the term “polypeptide” is intended to refer to a peptide or polypeptide of two or more amino acids. The term “polypeptide analog” includes any polypeptide having an amino acid sequence substantially the same as a sequence specifically described herein in which one or more residues have been conservatively substituted with a functionally similar residue and which displays the ability to functionally mimic an tissue factor polypeptide, as described herein. A “modification” of a tissue factor polypeptide also encompasses conservative substitutions of a tissue factor polypeptide amino acid sequence. Conservative substitutions of encoded amino acids include, for example, amino acids that belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His). Other minor modifications are included within tissue factor polypeptides so long as the polypeptide retains some or all of the structural and/or functional characteristics of an tissue factor polypeptide. Exemplary structural characteristics include sequence identity or substantial similarity, antibody reactivity, and presence of conserved structural domains such as RNA binding domains or acidic domains.

As with a tissue factor polypeptide, the invention also provides a functional derivative of a human tissue factor polypeptide. The term “functional”, when used herein as a modifier of a tissue factor polypeptide, or polypeptide fragment thereof, refers to a polypeptide that exhibits functional characteristics similar to tissue factor polypeptide. An exemplary functional characteristics of tissue factor polypeptide includes its procoagulant activity. One skilled in the art can readily determine whether a polypeptide, or encoding nucleic acid sequence, is substantially the same as a reference sequence by comparing functional characteristics of the encoded polypeptides to a reference tissue factor polypeptide.

The mutant non-human mammals can be produced by creating transgenic animals expressing a cDNA encoding a tissue factor polypeptide using a variety of techniques. Examples of such techniques include the insertion of normal or mutant versions of nucleic acids encoding a tissue factor polypeptide by microinjection, retroviral infection or other means well known to those skilled in the art, into appropriate fertilized embryos to produce a transgenic animal (Hogan et al., Manipulating the Mouse Embryo: A Laboratory Manual, Cold Spring Harbor Laboratory (1986), and U.S. Pat. Nos. 5,616,491 and 5,750,826), as described below in more detail.

A transgenic non-human mammal of the invention can exhibit a human tissue factor phenotype and is useful as an animal model of tissue factor related disease. As used herein, a “human tissue factor phenotype” refers to a functional or morphological change associated with a human tissue factor disease. A functional change associated with a human tissue factor phenotype includes any of a variety of changes associated with human tissue factor. Such changes can include, for example in a mouse model, procoagulation and clotting times.

A morphological change associated with a human tissue factor phenotype includes any of a variety of structural, anatomic or histological changes. Such morphological changes can include changes in protein expression in particular cell types, changes in the number or size of particular cells or particular cell structures.

A DNA fragment encoding a tissue factor polypeptide can be integrated into the genome of the transgenic animal by any standard method well known to those skilled in the art. Any of a variety of techniques known in the art can be used to introduce the transgene into animals to produce the founder lines of transgenic animals (see, for example, Hogan et al., Manipulating the Mouse Embryo: A Laboratory Manual Cold Spring Harbor Laboratory (1986); Hogan et al., Manipulating the Mouse Embryo: A Laboratory Manual, second ed., Cold Spring Harbor Laboratory (1994), U.S. Pat. Nos. 5,602,299; 5,175,384, 6,066,778; and 6,037,521). Such techniques include, but are not limited to, pronuclear microinjection (U.S. Pat. No. 4,873,191); retrovirus mediated gene transfer into germ lines (Van der Putten et al., Proc. Natl. Acad. Sci. USA 82:6148-6152 (1985)); gene targeting in embryonic stem cells (Thompson et al., Cell 56:313-321 (1989)); or electroporation of embryos (Lo, Mol Cell. Biol. 3:1803-1814 (1983)).

For example, embryonal cells at various developmental stages can be used to introduce transgenes for the production of transgenic animals. Different methods are used depending on the stage of development of the embryonal cell. The zygote is a good target for micro-injection, and methods of microinjecting zygotes are well known too (see U.S. Pat. No. 4,873,191). In the mouse, the male pronucleus reaches the size of approximately 20 micrometers in diameter which allows reproducible injection of 1-2 picoliters (p1) of DNA solution. The use of zygotes as a target for gene transfer has a major advantage in that in most cases the injected DNA will be incorporated into the host genome before the first cleavage (Brinster, et al. Proc. Natl. Acad. Sci. USA 82:4438-4442 (1985)). As a consequence, all cells of the transgenic non-human animal will carry the incorporated transgene. This will in general also be reflected in the efficient transmission of the transgene to offspring of the founder since 50% of the germ cells will harbor the transgene.

The transgenic animals of the present invention can also be generated by introduction of the targeting vectors into embryonal stem (ES) cells. ES cells are obtained by culturing pre-implantation embryos in vitro under appropriate conditions (Evans et al., Nature 292:154-156(1981); Bradley et al., Nature 309:255-258 (1984); Gossler et al., Proc. Natl. Acad. Sci. USA 83:9065-9069 (1986); and Robertson et al., Nature 322:445-448 (1986)). Transgenes can be efficiently introduced into the ES cells by DNA transfection using a variety of methods known to the art including electroporation, calcium phosphate co-precipitation, protoplast or spheroplast fusion, lipofection and DEAE-dextran-mediated transfection. Transgenes can also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (reviewed in Jaenisch, Science 240:1468-1474 (1988)). Prior to the introduction of transfected ES cells into the blastocoel, the transfected ES cells can be subjected to various selection protocols to enrich for ES cells that have integrated the transgene if the transgene provides a means for such selection. Alternatively, PCR can be used to screen for ES cells that have integrated the transgene. This technique obviates the need for growth of the transfected ES cells under appropriate selective conditions prior to transfer into the blastocoel.

In addition, retroviral infection can also be used to introduce transgenes into a non-human animal. The developing non-human embryo can be cultured in vitro to the blastocyst stage. During this time, the blastomeres can be targets for retroviral infection (Janenich Proc. Natl. Acad. Sci. USA 73:1260-1264 (1976)). Efficient infection of the blastomeres is obtained by enzymatic treatment to remove the zona pellucida (Hogan et al., supra, 1986). The viral vector system used to introduce the transgene is typically a replication-defective retrovirus carrying the transgene (Jahner et al., Proc. Natl. Acad Sci. USA 82:6927-6931 (1985); Van der Putten, et al. Proc. Natl. Acad Sci. USA 82:6148-6152 (1985)). Transfection is easily and efficiently obtained by culturing the blastomeres on a monolayer of virus-producing cells (Van der Putten, supra, 1985;

Stewart et al., EMBO J. 6:383-388 (1987)). Alternatively, infection can be performed at a later stage. Virus or virus-producing cells can be injected into the blastocoele (Jahner D. et al., Nature 298:623-628 (1982)). Most of the founders will be mosaic for the transgene since incorporation occurs only in a subset of cells which form the transgenic animal. Further, the founder can contain various retroviral insertions of the transgene at different positions in the genome, which generally will segregate in the offspring. In addition, it is also possible to introduce transgenes into the germline by intrauterine retroviral infection of the midgestation embryo (Jahner et al., supra, 1982). Additional means of using retroviruses or retroviral vectors to create transgenic animals known to the art involves the micro-injection of retroviral particles or mitomycin C-treated cells producing retrovirus into the perivitelline space of fertilized eggs or early embryos (WO 90/08832 (1990); Haskell and Bowen Mol. Reprod. Dev. 40:386 (1995)).

A DNA fragment comprising a cDNA encoding a human tissue factor polypeptide can be microinjected into pronuclei of single-cell embryos in non-human mammals such as a mouse. The injected embryos are transplanted to the oviducts/uteri of pseudopregnant females and finally transgenic animals are obtained.

Once the founder animals are produced, they can be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic mice to produce mice homozygous for a given integration site in order to both augment expression and eliminate the need for screening of animals by DNA analysis; crossing of separate homozygous lines to produce compound heterozygous or homozygous lines; breeding animals to different inbred genetic backgrounds so as to examine effects of modifying alleles on expression of the transgene and the neuropathological effects of expression.

The present invention provides transgenic non-human mammals that carry the transgene in all their cells, as well as animals that carry the transgene in some, but not all their cells, that is, mosaic animals. The transgene can be integrated as a single transgene or in concatamers, for example, head-to-head tandems or head-to-tail tandems.

The transgenic animals are screened and evaluated to select those animals having a human tissue factor phenotype, which are animal models for human tissue factor disease. Initial screening can be performed using, for example, Southern blot analysis or PCR techniques to analyze animal tissues to verify that integration of the transgene has taken place. The level of mRNA expression of the transgene in the tissues of the transgenic animals can also be assessed using techniques which include, but are not limited to, Northern blot analysis of tissue samples obtained from the animal, in situ hybridization analysis, and reverse transcriptase-PCR (rt-PCR). Samples of brain or other suitable tissues can be evaluated immunocytochemically using antibodies specific for an tissue factor polypeptide or a tag such as GFP. The transgenic non-human mammals can be further characterized to identify those animals having a human tissue factor phenotype useful in methods of the invention. In particular, transgenic non-human mammals expressing tissue factor polypeptides can be screened for a human tissue factor phenotype using the methods disclosed herein.

The invention additionally provides cells isolated from an invention transgenic non-human mammal. For example, the invention provides isolated mouse cells derived from an invention transgenic mouse. Cells derived from an invention transgenic.non-human mammal can be used, for example, in methods of identifying a therapeutic agent for treating neurodegenerative disease, as described in more detail below.

The invention additionally provides a DNA construct comprising a nucleic acid encoding a human tissue factor polypeptide. The term “nucleic acid”, also referred to as polynucleotides, encompasses ribonucleic acid (RNA) or deoxyribonucleic acid (DNA), probes, oligonucleotides, and primers and can be single stranded or double stranded. DNA can be either complementary DNA (cDNA) or genomic DNA, and can represent the sense strand, the anti-sense strand or both. Examples of nucleic acids are RNA, cDNA, or isolated genomic DNA encoding an tissue factor polypeptide. When expressed in a transgenic animal, the DNA construct comprises a transgene.

The DNA construct can comprise the nucleotide sequence of human cDNA encoding human tissue factor referenced as SEQ ID NO 2, to generate a human tissue factor polypeptide referenced above. The DNA construct can also comprise the nucleotide sequence of the mouse tissue factor gene as shown in SEQ ID NO. 3, where the mouse tissue factor cDNA is replaced by the human tissue factor cDNA. For example, in SEQ ID NO. 3, the region from the ATG in exon 1 through exon 2 of the mouse gene (nt 7,777 to 9,153 of SEQ. ID No. 3) may be replaced with the human tissue factor cDNA. The humanized TF allele sequence thus created is shown in SEQ ID NO. 4.

The nucleic acid encoding tissue factor polypeptide can be the same or substantially the same as a reference nucleic acid sequence, so long as the encoded tissue factor polypeptide exhibits structural and/or functional characteristics of a tissue factor polypeptide. Exemplary structural characteristics include sequence identity or substantial similarity, antibody reactivity, and presence of conserved structural domains such as RNA binding domains or acidic domains. Exemplary functional characteristics of an tissue factor polypeptide include RNA binding, transport of molecules, and/or exhibiting a potential human tissue factor phenotype. One skilled in the art can readily determine whether a nucleic acid sequence is substantially the same as a reference sequence by comparing functional characteristics of the encoded polypeptides to a reference tissue factor polypeptide.

As employed herein, the term “substantially the same nucleotide sequence” refers to DNA having sufficient identity to the reference polynucleotide, such that it will hybridize to the reference nucleotide under moderately stringent, or higher stringency, hybridization conditions. DNA having “substantially the same nucleotide sequence” as the reference nucleotide sequence can have at least 60%, at least 70%, at least 80%, at least 90%, or at least 95% identity with respect to the reference nucleotide sequence.

A nucleic acid can also include a modification of the cDNA referenced herein. As used herein, a “modification” of a nucleic acid can also include one or several nucleotide additions, deletions, or substitutions with respect to a reference sequence. A modification of a nucleic acid can include substitutions that do not change the encoded amino acid sequence due to the degeneracy of the genetic code. Such modifications can correspond to variations that are made deliberately, or which occur as mutations during nucleic acid replication.

Exemplary modifications of the sequences include sequences that correspond to homologs of other species, including mammalian species such as mouse, primates, including monkey and baboon, rat, rabbit, bovine, porcine, ovine, canine, feline, or other animal species. The corresponding sequences of non-human species can be determined by methods known in the art, such as by PCR or by screening genomic, cDNA or expression libraries.

The phrase “moderately stringent hybridization” refers to conditions that permit target-nucleic acid to bind a complementary nucleic acid. The hybridized nucleic acids will generally have at least about 60% identity, at least about 75% identity, more at least about 85% identity; at least about 90% identity; or at least about 95% identity. Moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 42° C. High stringency hybridization refers to conditions that permit hybridization of only those nucleic acid sequences that form stable hybrids in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5× Denhart's solution, 5×SPE, 0.2% SDS at 42° C., followed by washing in 0.1×SSPE, and 0.1% SDS at 65° C. Low stringency hybridization refers to conditions equivalent to hybridization in 10% formamide, 5× Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone,-and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); Ausubel et al. (Current Protocols in Molecular Biology (Supplement 47), John Wiley & Sons, New York (1999)).

The invention provides a DNA construct comprising nucleic acid encoding a human tissue factor polypeptide. As used herein, the term “DNA construct” refers to a specific arrangement of genetic elements in a DNA molecule. In addition to human tissue factor cDNA, or mutant forms thereof, the invention also provides for DNA constructs which contain other elements which are operationally linked. For example, the invention provides a DNA construct comprising nucleic acid encoding a tissue factor polypeptide operationally linked to a cell-specific expression element. The invention additionally provides green fluorescent protein (GFP) constructs.

DNA constructs of the invention can be incorporated into vectors for propagation or transfection into appropriate cells to generate invention mutant non-human mammals. One skilled in the art can select a vector based on desired properties, for example, for production of a vector in a particular cell such as a mammalian cell or a bacterial cell. If desired, the DNA constructs can be engineered to be operably linked to appropriate expression elements such as promoters or enhancers to allow expression of a genetic element in the DNA construct in an appropriate cell or tissue, for example, the murine tissue factor promoter (see Example 1).

The invention also provides vectors containing a DNA construct encoding a human tissue factor polypeptide. Suitable expression vectors are well-known in the art and include vectors capable of expressing nucleic acid operatively linked to a regulatory sequence or element such as a promoter region or enhancer region that is capable of regulating expression of such nucleic acid. Appropriate expression vectors include those that are replicable in eukaryotic cells and/or prokaryotic cells and those that remain episomal or those which integrate into the host cell genome.

If desired, the vector can contain a selectable marker. As used herein, a “selectable marker” refers to a genetic element that provides a selectable phenotype to a cell in which the selectable marker has been introduced. A selectable marker is generally a gene whose gene product provides resistance to an agent that inhibits cell growth or kills a cell. A variety of selectable markers can be used in the DNA constructs of the invention, including, for example, Neo, Hyg, hisD, Gpt and Ble genes, as described, for example in Ausubel et al. (Current Protocols in Molecular Biology (Supplement 47), John Wiley & Sons, New York (1999)) and U.S. Pat. No. 5,981,830. Drugs useful for selecting for the presence of a selectable marker includes, for example, G418 for Neo, hygromycin for Hyg, histidinol for hisD, xanthine for Gpt, and bleomycin for Ble (see Ausubel et al., supra, (1999); U.S. Pat. No. 5,981,830). DNA constructs of the invention can incorporate a positive selectable marker, a negative selectable marker, or both (see, for example, U.S. Pat. No. 5,981,830).

When a DNA construct is used to disrupt the expression of an endogenous gene in an animal by homologous recombination, the homologous sequence can be chosen from any genomic sequence so long as recombination of the endogenous gene with the homologous region in the DNA construct leads to disruption of the endogenous gene. In particular, the homologous sequence can contain an exon. The DNA construct is inserted into a cell and integrates with the genomic DNA of the cell in such a position so as to prevent or interrupt transcription of the native DNA sequence. When used to disrupt the expression of an endogenous gene in an animal, the DNA construct will generally contain an insert in the homologous region.

The invention additionally provides a cell comprising a DNA construct of the invention. For example, the cell can be an embryonic stem cell. As used herein, an “embryonic stem cell” is pluripotent stem cell derived from an embryo of a cognate organism for which introduction of a transgene is desired. In particular, the invention provides an embryonic stem cell comprising a DNA construct comprising a nucleic acid encoding an tissue factor polypeptide. Methods of using embryonic stem cells to generate a mutant non-human mammal are well known to those skilled in the art, as disclosed herein. For generation of a mutant mouse, embryonic stem cells can be obtained from a mouse. Alternatively, an appropriate embryonic stem cell line can be used to introduce a DNA construct of the invention.

The invention further provides an isolated cell containing a DNA construct of the invention. The DNA construct can be introduced into a cell by any of the well known transfection methods (Sambrook et al., Molecular Cloning: A Laboratory Manual, 2nd ed., Cold Spring Harbor Press, Plainview, N.Y. (1989); Ausubel et al., supra, (1999)). Alternatively, the cell can be obtained by isolating a cell from a mutant non-human mammal of the invention and establishing primary cultures. Thus, the invention provides a cell isolated from a mutant non-human mammal of the invention, in particular, a human tissue factor mutant mouse. The cells can be obtained from a homozygous mutant mouse or a heterozygous mutant non-human mammal such as a mouse.

The mutant non-human mammal of the invention is particularly useful as a model for human tissue factor disease, particularly for a disease having a common pathogenic mechanism as human tissue factor. Exemplary disorders include vascular disease, sepsis and cancer. As a model of various human tissue factor diseases, the invention mutant non-human mammal is useful for identifying a therapeutic agent for treatment of neurodegenerative disease.

The mutant non-human mammal of the invention can be advantageously used to screen for therapeutic agents that can be used to treat a human tissue factor disease. The invention thus provides a method of identifying a therapeutic agent for use in treating a tissue factor related disease by administering a compound to a mutant non-human mammal and screening the mutant non-human mammal for an improved response associated with a human tissue factor phenotype of the transgenic non-human mammal, thereby identifying a therapeutic agent for use in treating the human tissue factor related disease.

As used herein, “improved human tissue factor response” refers to any change in human tissue factor phenotype that is expected to result in a less severe human tissue factor phenotype. Such changes include improved functional and morphological changes associated with a human tissue factor phenotype. Improved functional changes include procoagulation and clotting times, and the like.

If desired, appropriate control animals can be used to corroborate the therapeutic effectiveness of screened compounds. For example, a control animal can be one that is not expressing tissue factor or a human tissue factor transgene.

Compounds useful as potential therapeutic agents can be generated by methods well known to those skilled in the art, for example, well known methods for producing pluralities of compounds, including chemical or biological molecules such as simple or complex organic molecules, metal-containing compounds, carbohydrates, peptides, proteins, peptidomimetics, glycoproteins, lipoproteins, nucleic acids, antibodies, and the like; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources. Combinatorial libraries of molecules can be prepared using well known combinatorial chemistry methods (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)).

Compounds identified as therapeutic agents by methods of the invention can be administered to an individual, for example, to alleviate a sign or symptom associated with a human tissue factor related disease. One skilled in the art will know or can readily determine the alleviation of a sign or symptom associated with a human tissue factor disease.

For use as a therapeutic agent, the compound can be formulated with a pharmaceutically acceptable carrier to produce a pharmaceutical composition, which can be administered to the individual, which can be a human or other mammal. A pharmaceutically acceptable carrier can be, for example, water, sodium phosphate buffer, phosphate buffered saline, normal saline or Ringer's solution or other physiologically buffered saline, or other solvent or vehicle such as a glycol, glycerol, an oil such as olive oil or an injectable organic ester. A pharmaceutically acceptable carrier can also contain physiologically acceptable compounds that act, for example, to stabilize or increase the absorption of the modulatory compound. One skilled in the art would know that the choice of a pharmaceutically acceptable carrier, including a physiologically acceptable compound, depends, for example, on the route of administration of the composition.

The methods of the invention can advantageously use cells isolated from a homozygous or heterozygous mutant non-human mammal, for example, epithelial cells, nerve cells, fibroblasts, muscle cells, or any appropriate cell isolated from a mutant non-human of the invention for a desired purpose. The methods of the invention can also be used with cells expressing DNA encoding a tissue factor polypeptide such as a transfected cell line.

A cell expressing a DNA construct encoding a tissue factor polypeptide can be used as an in vitro method to screen compounds as potential therapeutic agents for treating human tissue factor disease. In such a method, a compound is contacted with a cell expressing human tissue factor, either a transfected cell or a cell derived from a human tissue factor mutant non-human mammal, and screened for alterations in a phenotype associated with expression of human tissue factor.

A tissue factor fusion polypeptide such as tissue factor-GFP can be particularly useful for such screening methods since the aggregation can be monitored by fluoresence intensity. Other exemplary fusion polypeptides include other fluorescent proteins, or modifications thereof, glutathione S transferase (GST), maltose binding protein, poly His, and the like, or any type of epitope tag. Such fusion polypeptides can be detected, for example, using antibodies specific to the fusion polypeptides. The fusion polypeptides can be an entire polypeptide or a functional portion thereof so long as the functional portion retains desired properties, for example, antibody binding activity of fluorescence activity.

The invention further provides a method of identifying a potential therapeutic agent for use in treating a human tissue factor disease. The method includes the steps of contacting a cell containing a DNA construct comprising nucleic acid encoding a tissue factor polypeptide with a compound; and screening the cell for an improved human tissue factor phenotype, thereby identifying a potential therapeutic agent for use in treating a tissue factor related disease. The cell can be isolated from a transgenic non-human mammal having nucleated cells containing the DNA construct. Alternatively, the cell can contain a DNA construct comprising a nucleic acid encoding a green fluorescent protein fusion, or other fusion polypeptide, with an tissue factor polypeptide.

Animal Models Useful for Pre-clinical Testing

In addition to their ability to be used for screening, the mutant non-human animals of the invention are useful as animal models for use in pre-clinical testing. In pharmaceutical research, it has become commonplace to perform as much efficacy testing as well as safety testing in animals as possible prior to subjecting human subjects to the dangers of exposure of a novel compound or biologic drug candidate. In the field of biopharmaceuticals, the human or “host” response to the test agent can include immune reaction to the protein which is an as yet unpredictable event, but also a myriad of other similarly complex effects. For example, while TNFalpha is capable of killing tumor cells in vitro, injection of TNFalpha into a living mammal precipitates a “cytokine storm” which can be lethal. Thus, it is important to understand not only the direct effects of therapy but the host response to it as well.

Commonly used animals for testing are mice, rats, guinea pigs, dogs, cynomolgous monkeys, and nonhuman primates. Some disease states have animal analogues either due to a natural or genetic defect. One example is the naturally obese and diabetic mouse genetically designated db/db. In other cases, models of disease can be induced in otherwise health animals; such as collagen-induced arthritis in mice or dextran induced inflammatory bowel disease. Although xenografts models have been developed whereby cells or tissues of one species are grafted into the bodies of another species, for example human tumor xenografts in mice, the host animal must be chemically or genetically immunosuppressed in order not to reject the tissue. Secondly, xenografts models may suffer from the complication that the target ligand is produced by the graft while its homolog protein is produced by the host.

While wishing to remain within humane guidelines and minimize the use and suffering brought to bear on any living entity, nonhuman testing as well as human testing is often necessary and indeed required for approval of new therapeutic products.

Biopharmaceuticals and, particularly, antibodies interact with a high degree of specificity on their targets as the interactive “face” is on the order of several hundred angstroms. For this reason, slight changes in the target, especially surface charge, can alter specificity and affinity of binding. Across species, especially within the family mammalae, there are many highly conserved protein structures and substructures or domain. However, antibodies to a protein from one species of mammal often do not recognize the protein from another species that performs the analogous role and may have considerable sequence identify if there are changes in the residues that form the binding face with that antibody.

For this reason, an expedient method of selecting a biologic surrogate which functions in a nonhuman animal in a manner as close a possible to the human therapeutic candidate would be of great utility. The present invention thus provides a model for the evaluation in preclinical models for those biopharmaceuticals such as the TF8-5G9 antibody which do not bind to the endogeneous tissue factor such as murine tissue factor.

Having described the invention in general terms, it is further embodied by the following examples.

### EXAMPLE 1

### Generation of HuTF (h/h) Mice

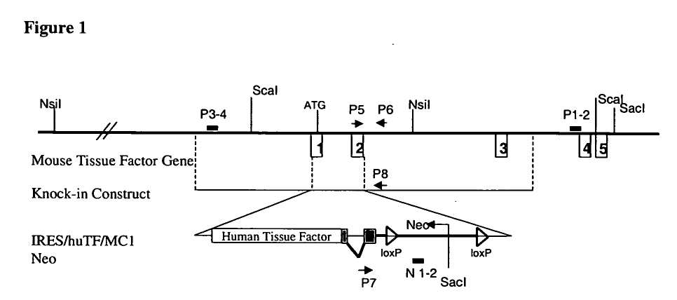

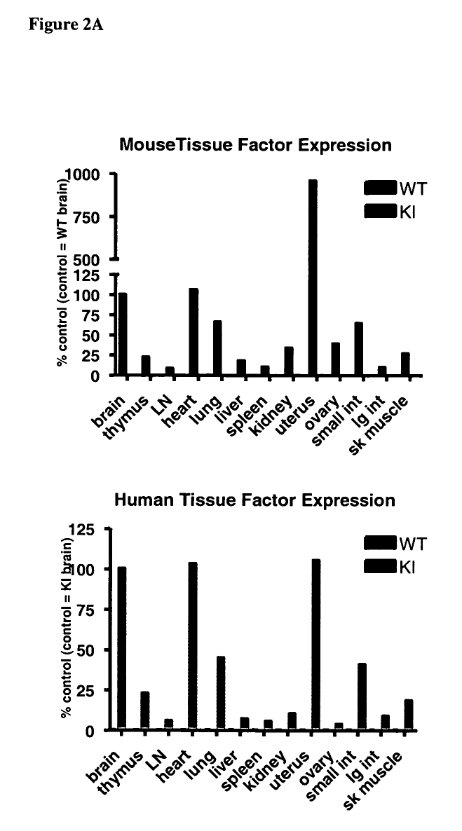

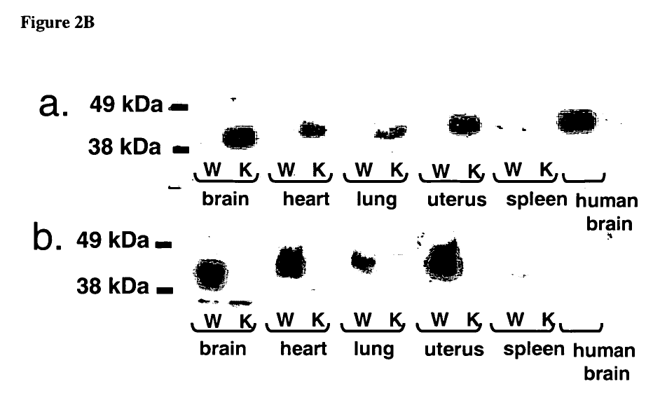

HuTF knock-in homozygous (h/h) mice were generated by homologous recombination in embryonic stem cells followed by intercrosses between heterozygous (h/m) mice. In order to generate mice that express huTF under the control of the endogenous muTF promoter, the open reading frame of huTF was placed in-frame at the initiation codon of the muTF gene, which replaced the first two exons of muTF (FIG. 1). Deletion of the first two exons of muTF gene has been shown to eliminate expression of muTF and result in mortality in utero (Bugge T. H. et al., 1996. Proc Natl. Acad. Sci USA 93: 6258-6263; Carmeliet P. et al., 1996. Nature 383: 73-75; Toomey J. R., et al., 1996. Blood 88: 1583-1587), though expression of just 1% huTF in the transgenic mice rescued them from death (Parry G. C. N. et al 1998 J. Clin. Invest. 101: 560-569 ). The (h/h) mice appeared to develop normally and were born according to Mendelian frequency. The (h/h) mice showed no obvious abnormalities in size, weight, fertility, or behavior and appeared healthy. These results indicate that the huTF knock-in gene was functionally replacing the missing muTF gene. Western blotting experiments showed that in (h/h) mice, huTF protein, but not muTF protein, was expressed (FIG. 2B, Example 5).

HuTF (huTF) knock-in mice were generated from the Protamine Cre ES cells by Lexicon Genetics Inc (Woodlands, Tex.). The Protamine-Cre ES cells allow excision of the Neo^{R} selection cassette. In this ES cell line, Cre is expressed under the control of the Protamine promoter during spermatogenesis. Thus, when the chimeric mice generated from this ES cell line are bred, the targeted allele passes through the male germline and the Neo cassette, which is flanked by loxP sites, is excised. The resulting heterozygous mice contain the humanized tissue factor allele without the Neo^{R} selection cassette. Briefly, a genomic fragment from a 129SvEvBrd DNA clone containing the mouse TF gene was used to generate the targeting construct. The genomic fragment containing exon 1 and 2 of mouse TF (muTF) was deleted and replaced by an expression cassette IRES/huTF/MCl Neo (FIG. 1). The open reading frame of huTF was derived from the image clone 4073183 and inserted in-frame at the initiation codon of muTF gene, to enable transcription of huTF to be driven by the muTF promoter. The neomycin resistance gene (Neo) was included for positive selection of homologous recombinants, and the HSV-thymidine kinase gene was included for negative selection against random integration. The targeting construct was transfected into 129SvEvBrd embryonic stem cells and cells were cultured in the presence of 400 μg/ml G418 and 0.2 μM ganciclovir. Homologous recombination events were confirmed by Southern blot analyses. Chimeric mice derived from targeted ES clones were generated by standard methods and bred with C57Bl/6 mice to produce huTF knock-in heterozygotes (h/m) to produce the N1 generation. Further backcrossing with C57Bl/6 mice was performed to generate a breeding colony of huTF h/m (N2). For characterization studies, huTFKI homozygous (h/h) mice and wild type (m/m) littermates were derived from breeding the N2 heterozygous mice.

Southern Blot Analyses:

Homologous recombination events in the ES clones were identified by digesting genomic DNA with ScaI and probing with huTF genomic DNA probe P1-2 (FIG. 1). P1-2 is outside the targeting construct and was generated by PCR amplification of genomic DNA using primer P1 5′-CTGGAGCTGATTCTGTACAG and primer P2 5′-CTGGAATCTCTGAAAGGACC. The targeted allele was identified as an 13.8 kb band, while the wild type allele was identified as an 11.5 kb band. The specificity of the homologous recombination event was confirmed by Nsi digestion using a 5′ probe P34 (FIG. 1). P34 is located inside the targeting construct and recognized a wild type allele of 6.8 kb and a targeted allele of 9.0 kb.

To verify single site integration in targeted ES clones, genomic DNA was digested with SacI or ScaI and probed with a Neo-specific probe Neo1-2 (FIG. 1). A single band of 10.7 kb for SacI or of 13.8 kb for ScaI was detected in selected ES clones, indicating that no random integrations of the targeting vector were present.

PCR Genotyping

To distinguish the wild type and targeted alleles, tail DNA was PCR amplified. A 480 bp product of wild type allele was identified using primer P5 5′-GTAGGCATTCCAGAGAAAGC and primer P6 5′-CTGGAACTCCCTATGTACAG. A 267 bp product of the targeted allele was identified using P7 primer 5′- GTAGCCAGCAGATTACCATG and P8 primer 5′5′-GTTGAGATGGGACTGCAGGAA.

### EXAMPLE 2

### Tissue Distribution Of HuTF In (h/h) Mice

Data from RT-PCR and Western blot analyses demonstrate that huTF expression in the (h/h) mice is comparable to that of muTF in the (m/m) mice (FIG. 2A, B). The real-time RT-PCR experiments demonstrate that for both (m/m) and (h/h) mice, TF was highly expressed in the brain, heart and uterus, modestly in the lung and small intestine, and at low levels in lymph node, liver, spleen, kidney, ovary, large intestine, and skeletal muscle. Western blotting experiments confirm that the RNA expression correlates with protein expression in the brain, heart, lung, uterus and spleen (FIG. 2B).

Tissue Harvest:

Tissues were harvested from male and female huTF (h/h) mice and their (m/m) littermates. Upon necropsy, all tissues were cut longitudinally and each half was snap frozen on dry ice and stored at −80° C. until subsequent RNA or protein isolation.

Quantitative Real Time RT-PCR Assay:

RNA was isolated from each snap-frozen tissue using the Trizol method according to the manufacturer's instructions (Invitrogen). After DNase treatment, RNA from each tissue was quantitated and pooled (3 animals/tissue) into four groups: (h/h) male, (h/h) female, (m/m) male and (m/m) female. Quantitative RT-PCR was performed on the Taqman 7900 Detection System (ABI, Foster City, Calif.). Amplification of specific PCR products was performed in a total of 25 uL containing 5 uL RNA template (50 ng/reaction), 5′ and 3′ primers, a dual-labeled probe, and 2X RT-PCR Master Mix (ABI). Dual labeled probes with 6-carboxy-fluoroscene (6FAM) at the 5′ end and 6-carboxy-tetramethylrhodamine (TAMRA), a quencher fluorescent dye at the 3′ end were used to detect murine and human TF. Primers and probes for human TF spanning intron 3 (forward, 5′ TCCCCAGAGTTCACACCTTACCT-3′; reverse, 5′-CACTTTTGTTCCCACCTGTTCA-3′; probe, 6FAM-AGACAAACCTCGGACAGCCAACAATTCA-TAMRA), murine TF spanning intron 1 (forward, 5′-CGCGCCCACGTTTCTC-3′; reverse, 5′-AATTAAACGCTTTCTCTGGAATGC-3′; probe, 6FAM-CCTCCAGGTGACCGCGGGT-TAMRA), and murine TF spanning intron 5 (forward, 5′-CCTGGCCACCATCTTTATCATC-3′, reverse, 5′-TGTCCCGCTCGGTTCTTTC-3′; probe, 6FAM-TCCTGTCCATATCTCTGTGCAAGC-TAMRA) were designed using Primer Express (ABI). PCR primers were designed to span an intron to avoid amplification of genomic DNA. As an endogenous control, 18S ribosomal RNA was amplified using a probe labeled with VIC at the 5′ end and 6TAMRA at the 3′ end (ABI). Reverse transcription was performed at 48° C. for 30 minutes. Amplifications were performed at 95° C. for 10 minutes and for 40 cycles of 30 seconds at 95° C. and 30 seconds at 60° C. Fold-induction for both endogenous control and gene of interest were calculated from relative standard curves generated from an internal sample known to express TF or 18S.

Protein Isolation and Western Blot Analysis:

Individual tissues were homogenized in M-PER reagent (Pierce) containing complete—EDTA free protease inhibitors, kept on ice, and centrifuged at 13,000 rpm for 10 minutes at 4° C. Supernatants were collected while cellular debris was pelleted and discarded. Lysates (50 μg) were separated on a 4-12% SDS-polyacrylamide gel under non-reducing conditions. Protein was transferred to nitrocellulose and blocked with 1X PBST containing 5% non-fat dry milk for 1 hour at room temperature. Nitrocellulose blots were probed overnight with goat anti-huTF antibody (American Diagnostica Inc.) or rabbit anti-muTF polyclonal antibody (a kind gift from Dr Jim Morrissey) at 4° C., followed by incubation with horseradish peroxidase-conjugated goat anti-rabbit or rabbit anti-goat secondary antibody (Jackson Immunoresearch). Immunoreactive bands were visualized using chemiluminescence (ECL Plus Western Blotting Detection System, Amersham).Individual tissues were homogenized in M-PER reagent (Pierce) containing complete—EDTA free protease inhibitors, kept on ice, and centrifuged at 13,000 rpm for 10 minutes at 4° C. Supernatants were collected while cellular debris was pelleted and discarded. Lysates (50 μg) were separated on a 4-12% SDS-polyacrylamide gel under non-reducing conditions. Protein was transferred to nitrocellulose and blocked with 1X PBST containing 5% non-fat dry milk for 1 hour at room temperature. Nitrocellulose blots were probed overnight with goat anti-huTF antibody (American Diagnostica Inc.) or rabbit anti-muTF polyclonal antibody at 4° C., followed by incubation with horseradish peroxidase-conjugated goat anti-rabbit or rabbit anti-goat secondary antibody (Jackson Immunoresearch). Immunoreactive bands were visualized using chemiluminescence (ECL Plus Western Blotting Detection System, Amersham).

### EXAMPLE 3

### Normal Coagulation Activity of HuTF from the (h/h) Mice

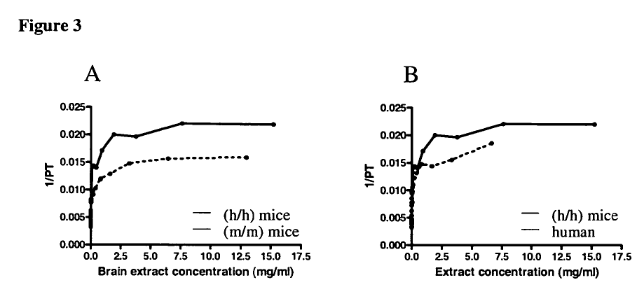

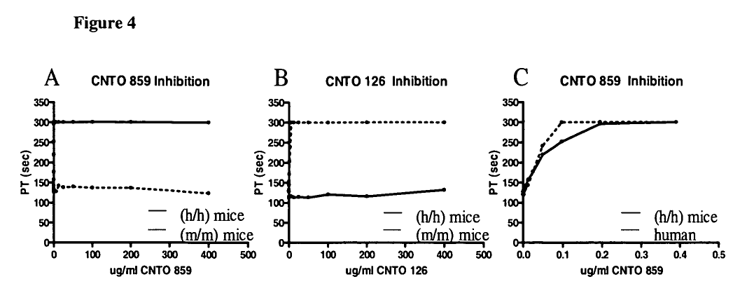

The huTF expressed in the (h/h) mice was functionally capable of replacing muTF, as shown by the ability of the (h/h) brain extract to initiate robust clot formation in the prothrombin time (PT) assay (FIG. 3). The (h/h) brain extract activity was very similar to that of the human brain extract or the (m/m) brain extract (FIG. 3). The coagulation activity from (h/h) brain extract was blocked selectively by a neutralizing anti-huTF antibody, CNT0859 (FIG. 4). The IC₅₀ of CNT0859 for the (h/h) brain extract was similar to that for the human brain extract, indicating comparable levels of TF activity from both samples (FIG. 4C). A mouse-specific anti-TF antibody PHD167 had no effect, supporting the conclusion that expression of the endogenous muTF is eliminated in the (h/h) mice. Consistent with previous reports showing the ability of huTF to reconstitute mouse coagulation (Parry G. C. N. et al., 1998 J. Clin.

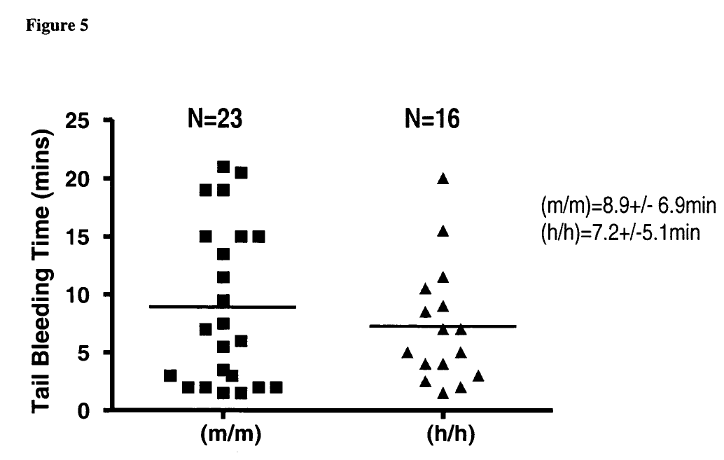

Invest. 101: 560-569), the (h/h) mice showed no apparent tendency for increased bleeding, either spontaneously or from surgical sites. The bleeding time for the (h/h) mice and the (n/m) mice were also similar (FIG. 5; 7.2±5.1 min, n=23, and 8.9±6.9 min, n=16, respectively).

Brain Homogenates for Source of TF: Brain homogenates were made from human brain, (m/m) mouse brain, and (h/h) mouse brain. Approximately 25 mg brain sections were homogenized in Hanks Balanced Salt Solution (HBSS) using Fast-prep protein isolator tubes (Bio 101 Systems, 6913-100). Brain homogenate was frozen at −80° C. immediately after isolation, and was diluted 1 to 100 immediately prior to use. The total protein was measured at OD₂₈₀ to determine the concentration.

TF Activity Assay: TF procoagulant activity in the brain homogenate was measured using a one-stage clotting assay. Citrated human plasma (100 μL) was incubated with increasing concentrations of brain homogenate (200 μL and 10 mM CaCl₂) and the clotting time was recorded using an Organon Teknika Coag-A-mate XM. All samples were tested in duplicate. The clotting time was plotted as a function of the brain homogenate and the data were fit to a hyperbolic curve to determine an EC₅₀.

TF Inhibition Assay: Brain extracts were diluted to their EC₅₀ in HBSS and 15 mM CaCl₂. Each extract was then incubated with increasing concentrations of either an anti-huTF mAb (CNTO 859) or an anti-muTF mAb (CNTO 126) or both, and pro-coagulant activity was measured with a one-stage clotting assay. All reagents were maintained at 37° C. Briefly, 200 ul extract with 15 mM CaCl₂ and mAb was incubated with 100 ul pooled citrated human plasma and the clotting time recorded using an Organon Teknika Coag-A-mate XM. All samples were tested in duplicate. The clotting time was plotted as a function of Ab concentration.

Tail bleeding assay: The tail bleeding time was measured according to the method by Dejana (Dejana EA, 1979 Thrombosis Res. 15:191). Briefly, 2-3 month old (m/m) or (h/h) littermates were placed in a restraining chamber and 1 cm of the tail tip was amputated. Emerging blood from the tail tip was blotted gently onto a filter paper every 15 sec until complete occlusion of the vessel was achieved. The total bleeding time of each animal was calculated based on the number of blood spots blotted. No animal bled for more than 30 min in this study.

1. A transgenic non-human animal comprising cells containing a DNA sequence encoding a human tissue factor, said sequence operatively linked to a promoter endogenous to the non-human animal, wherein said non-human animal exhibits a human tissue factor phenotype.

2. The transgenic non human animal of claim 1, wherein said non human animal is a mouse.

3. The transgenic mouse of claim 2, wherein said mouse is homozygous for said transgene.

4. The transgenic mouse of claim 1, wherein said mouse is heterozygous for said transgene.

5. The transgenic mouse of claim 1 wherein the mouse does not express endogenous murine tissue factor.

6. A method of identifying a therapeutic agent for use in treating a tissue factor-related disease, comprising:
(a) administering a compound to the transgenic mouse of claim 1, and
(b) screening said transgenic mouse for an improved response associated with a human tissue factor phenotype of said transgenic mouse, thereby identifying a therapeutic agent for use in treating said tissue factor related disease.

7. The method of claim 6, wherein said mouse is homozygous for said transgene.

8. The method of claim 6, wherein said mouse is heterozygous for said transgene.

9. A cell isolated from the transgenic mouse of claim 1, wherein said cell expresses said human tissue factor polypeptide.

10. The mouse cell of claim 9, wherein said mouse is homozygous for said transgene.

11. The mouse cell of claim 9 wherein said mouse is heterozygous for said transgene.

12. A method of identifying a potential therapeutic agent for use in treating a tissue factor related disease, comprising:
(a) contacting a cell of claim 9, said cell containing a DNA construct comprising a DNA sequence encoding a tissue factor polypeptide, with a compound, and
(b) screening said cell to identify a compound having activity that alters a phenotype associated with human tissue factor polypeptide expression, thereby identifying a potential therapeutic agent for use in treating said tissue factor related disease.

13. The method of claim 12, wherein said DNA construct comprises a DNA sequence encoding a green fluorescent protein fusion with said human tissue factor polypeptide.

14. A transgenic mouse comprising cells containing a DNA sequence encoding a human tissue factor, said sequence operatively linked to a murine tissue factor promoter.

15. The transgenic mouse of claim 14, wherein said mouse is homozygous for said transgene.

16. The transgenic mouse of claim 14, wherein said mouse is heterozygous for said transgene.

17. A method of identifying a therapeutic agent for use in treating a human tissue factor disease, comprising:
(a) administering a compound to the transgenic mouse of claim 13 and
(b) screening said transgenic mouse for an improved response associated with a human tissue factor phenotype of said transgenic mouse, thereby identifying a therapeutic agent for use in treating said human tissue factor disease.

18. The method of claim 17 wherein said mouse is homozygous for said transgene.

19. The method of claim 17 wherein said mouse is heterozygous for said transgene.

20. A cell isolated from the transgenic mouse of claim 14, wherein said cell expresses a human tissue factor polypeptide.

21. A method of identifying a potential therapeutic agent for use in treating a human tissue
factor disease, comprising:
(a) contacting a cell of claim 20, said cell containing a DNA construct comprising a DNA sequence encoding human tissue factor polypeptide, with a compound, and
(b) screening said cell to identify a compound having activity that alters a phenotype associated with human tissue factor polypeptide expression, thereby identifying a potential therapeutic agent for use in treating said human tissue factor disease.

22. A DNA construct comprising a DNA sequence encoding a human tissue factor polypeptide, operationally linked to a promoter element.

23. A vector comprising the DNA construct of claim 22.

24. An isolated mouse cell comprising the DNA construct of claim 22.

25. A DNA construct comprising a DNA sequence encoding a fusion polypeptide of human tissue factor polypeptide and green fluorescent protein, operationally linked to a cell-expression element.

26. The DNA construct of claim 25, wherein said human tissue factor polypeptide comprises the amino acid sequence referenced as SEQ ID NO. 1.

27. A mouse embryonic stem cell containing a DNA construct comprising a DNA sequence encoding a human tissue factor polypeptide, said sequence operatively linked to a promoter, and said human tissue factor polypeptide expressed in said embryonic stem cell.


SEQUENCE LISTING (4 sequences)

SEQ ID NO: 1 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 2 - DNA, 1374 bp, Homo sapiens
gggggcgccgccggccctttatagcgcgcggggcaccggctccccaagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacaaatggtactacaaccaattccaagttttaatttttaacaccatggcaccttttgcacataacatgctttagattatatattccgccgtcccaaaaaaaaaaaaaaaaaaaaaaaaaaaaaa

SEQ ID NO: 3 - DNA, 21694 bp, Mus musculus
  unsure (3465)(3466)(3467)(3468)(3469)(3470)(3471)(3472)(3473)3474)
  (3475)(3476)(3477)(3478)(3479)(3480)(3481)(3482): Where n can equal a, c, t, or g
ctaccctgtcccatgaggccagagagaaggctaagaccaggtaagactgttgttccaagggttcagttcccagcactcccattgggtggctcacaaccatctgtatctgcaccttcagagactccaaccccttgtctcttcaggtccccacacacatgtcagacaggttcactctctccctagcctctaacacacacacacacacacacacacacacacacacacacacacacacacacacacagagggaggataaaaagaaatcctttaacttgtcccagaggcaggctctgaggtcaccactgggaagagcacactaagtttatggaagtgagtggcttggggagccaggtcagtgctggggccactggggcatctcacaggagtaagcttcttgggggagactccctctggagctctccgggagggagggaccgctgaacagacagagccaagcaagtgcaggtgattccatcacaagtcccatcttgagccagggtggtggtgtcatgatgataaacagcagacaggcaagaaatgtgctgcggaatggcaggatgataagggggaactgattgcaatcaagcacactcatcagtttccttagcaagtctgcaaggcctttcctaccccaggttgtgagttggaagggtgacccatatttctcgatgaatgacagaccctgctgcgggacacggatgccatgttcttgaggcaaataagccattttcagttacaatatctttcttcttctctgttctgggtaggagtgcaacttgaatgtagaaacaccagaacaaaataaactggaaacaaggcaaaagaaagagccctttcttctcaactaagaccagatttggcttggttttcttttggaaaacctccatattctgaagccaggcactctagagaggcacagctcacaaggtagggaacactgagggcagcagggacagggacttgaattcatgctgcaatttcttttaaaggatttatttgtttgtttatttatttatttacttatttattttatgtgagtacacacactgtcactttcttcaaacacaccagaagaaagcaccaggtcccattaaagatggttgtgagccaccacgtggttgctgggaattgaactcaggacctctggaagagcagtcagtgctcttaaccgctgatgcttctctccagcccccatgctgcaatttccacagtgctcccatgggaagtgtgtgttaccgcacattcagtgagacaggaagcactgtaagacagtaagacatagcactgcgattatgaagttgtactcaagggctccgatagtcagatgcaaccccagctcaccattatccagttgtgtgacttctgaacaacttgttcaactgcctttggcttcagttaacccatctgtaaattcatccctatctgtcaaaaatgggattactctgaggactaagtgacttaatacttggtggctaataaatggtattttctggctattttcaatgtcaggaagattatagcatggtcctgagattcactgagtttccaaagtttatctgcttttccactgggacatgatgtcagctaagaagaaatacaagagggaataaagttatttatagagttcctggattcttagaagatcttgcccatgacagaaaccatcgtaatcgtaattgtcatctgagccgcatttcattattgcctaaggccagctggtagcatgtgggaatttgggtgttgtatggtaatctcctttgaactaaaaaattccatgctggagagaagctcatgaatgctcaggaagtatttgtgtgtgtacatacatgtgtgcatgtttgtgtggatgtgtttgctacatgcagacaacgtcagtagaggccacaaaataagctgcctggtgtgggtgctaggaactgcaaatctctgtaagagcaacacgtgcccttaactgttgagccatctttccagtcccaacacttatagaaagttttaaaggtaggttatttaagattgaggaaataaacaactcagaaatctcagaaactcccccaaatttatcaaattaacaaggctcctcccttcccatggctttacatgcagtaagaactgctgaggggaggagactttctcacctgtcaagctggctacaggtcatacagagagctccaaggtcccagcttttgtgagttttcatttatgctgggtgggctttggtgatacaactgcctttaaatcattcctccttctacgagaactctttcatccatatttctctaagtaaccccataaaaactcatttgtttaccgagttggactttagtggcattgttactttgatctgttaccagttccctatctgaggtgagtaggcttttgttcgtatctcccccagaatggtttcacataactcggagtttctaggctttagagcagggataaaggcaaattcctagacaactgagtcttaggtcagagcgagctagactgaaaccatgagtaaccttgatgatagagaaatcaaagtttggggaagttgatctgaatcactgatatgcatctctcaagtcagcagtagtttcctttcttgctttaacccaaagaagtgggaaacgtaacaagatccatgtgtcttttaggacctgttgtaaccacacatataactggattttaccaagtcaagtttcatggtcttctctttctgcaaatttcaatcctcgttccttctccagatcctcccaggtcacctcccgccctcctctagccacacaccaaggaccagcatcaagtcttgttcctgtccgttatggcttggatgaaaaatgtcccccacaggctggtgtatgaacatttggcatctcatccgtggtgctgttcagggtggatgtagggttatagaacacttaagaactagagctttgctgggggaagtgcatcactgaggagaggctttgagtatgtgtagcctcacccaacttccggttccctctgtctgcttcctgtgtgtagatgaaatgtgacctctctgattcctaacagccagttcgccgcctgttccggcttctgtgtcttccccagcgcaatggaccctatccctctggaactagaagcaaatacaaatcccttcttctttaagttgattttgtcaggttattttatcacaacctcaggaaagtaattgacacgccatctgtccagtagctttagcagtcctcttctctggtctcagaccccctctgcctccaaaataggtcacacagcccagacttggatgttcctctggcaactgagattgcccacggatggtcccagatcggagcagaaacaactggaatcttatagtagtactggtgattggctccagatgtgacaaagccaagacatgtattctgggctgttggtgacttctaggcactgttccatttgttctaagagcttcctgggctcgtaaactgtaactttttnnnnnnnnnnnnnnnnnnccacctcctccactcactgatctcagcccttgccctggcctttgctcttcctctgtgctccagtttaccatgatctgaatgttggtgccacctgaaaatgcatatgttgaaacctcatgcaggatacaatagtatcaagaggtgggaggggtttctggatgtgattaagttaaaaggctccgccctcataagttatatagaggatcaatgcccttatgagaaggccatggtgggtcactccctctcccctgctgtgaggacacaggtcaccatctttgaagcccagaatgtgcctttgagaaaccctgattctattgacacttttgtcttagacttctcaggctccagcactggggtctttgttactgcaaactctcctgccttattttcctccttcctcctctcctcccttctttttcccctgccctactttccttcttgtgtatagctgccaaagtgatcactgaagtattttgtaagagtggcctgaatggacaaagacgttgctcaaactctgctttcccccaaggaaccttctctgatgaactggtcagaagaaagttctccagggggtcccgagtagttttacgttataaacaaagctttaaggatatgagaacctgtcctgggaaacctgtgtattaccaaaaccagagcagtcactattagcatgaagttttcagtctatgagtaagacatgtatctcagacctcagttctgctccatccttgtaggaagtgtctatttgtttttctccctgacttataagctatttagagagagagagagagactcaaacatagttcctgtggtatattgtcccatacatagtaatccctcaataaatatttattaagttacacaaaccaagatcttcattattccctggtgataggaaagacaaacacttaactttttctctttttttttctttctttttttttctccctttccttggttggttggtttggttttttgagacagggtttctctgtgtagccctggctgtcttggaactttctttatagaccataccaggacagcctcgaacttagagtgctgatcgctaagataggcagcctactagccctgagtctgcacaggtacttatgtgctaaaaatcccaagacttagactggaagattgtcctctaaaatcatggggctttagattttcttcttaaagataggtgcagtggtgcatgcctttaatcccagcactcgggaggtaaaggcaggtggatctctctgagctggacacagcctggtctatactagtttcagtccagccagggagacataatgagaacttgtctcaaaacttaataataagtgaataagcagatagaaacagtgtttttcaaaatcttggtgctatgtgataactaagttagtggcctagcaactgtattttgaatacagtttttgaatggtcaggttaacgttatacaagttttcacctaattttggatgaaatataaactgttggatagtacataattattttcacaaaactcctgaagcacagcagccattttctccatgactaaccctgccagatgtttcttcggttttgctgagtcactttttctcccgactcagaactctgcgttctcactaactctgaagttccactggcattttctcatattttgccttttacctgaaaaaaaatgggaaggggggaatggttaaattgcttattttactatctcacacgcatataaccagttttttaaaaaaaaaataattactttcagttttaccttcggtcataaatcttaacctgtccttttcctgggaagacagacattttgaggaatttatgctaaacagcctttaagcctggctatcaacagtcacgttgcatcccatgactgagcagcttctgaatcaccctcgagtgtaagctgttagaaaaaaatgaaagcagcccccaaacgtcttcattctgttctgaaactacctcttctccaggtagaagtcctagaaaccggaaggaccctcagccaccttgagcgggctggggctggctggggagcaaagtccttggaatccataggtttgcatttcctcaccaatctgatttgctcactgcctggctcccagacagaagcgcttcagcgacaagagttcatctggtgaatgttaatgaaaactctcagcgacaagtgtggctgcataaatgaattttattgattcaaatgtgtcaagttcctcctgggtgcttgtaaactgtgctaagagctaaaggggggacctgatcccccctccccctaaaagagcttgatgggggaagttgacagagggcaccattgagattatgtaaagtacttgatgaacttggttggtaaagctgttagcatgctctataaacgtgcacaagaagggctaaaagcgtgccctggtcacttacagactcctcatcagccacattgcaccactggtgatgcaaaaggaaactgggctctctacgtgtcttcacgcatgtctgctttaaggaatagccaacaggatatttctgcctatgaacaataagttccccaaaccgcgagtgtttcccacacctgctgaggaacacagttgagggtccgtggatggaaaaaagatcaccctttgaacagttttcttgatgccctcttcctttgctcagttgggagtaaaagaaagtgcggtcaagagggaaagtccctggagtagaggaccagatagagctacattgtgtacttaggccagtgagcacacggaccagctgacctcaagattggtattgtagaaggctatctaagaggttctgccctgtccattctcagacataagcaggaagcctaatcaaaagctactgtgagtgggacgacagcttagttactctttacacttctcagagcctacaaggtggacccaagtttgactttactcaaggaaagtaaaaaaggaaggaagaaaggaaggaaggaaggaaggaaggaaggaaggaaggaagattgtacaaagcatccaaagacacagatatttacattacagttcataaccgtagcaaaattacagttatgaagtagcaataaaaataattttatggttgggatcactacaatatgaggagctgtgttaaagggtcgcagaattagggaaattggaaaagaagggataaactggacagtcaaccaggagtgacaagaaatcgtttgatacactgtcgagcaaatgctaccagtaggataagtgatcgtctaaggcttcaacaacgtcacggcaaagcctagaacaggaggccaggaaataaatgaagaaaacccaaagcttgcaagtgacacctggctgccactaggctcagtgacagaggtgggctcctgggtccattcccagcatccaggaagggaaaacacaaattggaacgtccgaatgtttatttttcagtttcattttttgtttttctgcgctttggaggaattcactcaaacccactgcgggcttcctcaacgtgagaatcatccttcctggaacccccgtggctacagaagtcacgctcaaattctctcaggcttcatgttgcagttcctcggtcacctcgggaacgggagtggggtgccaagtggcctttagacctaatccagggcccttgtccatggcagacacgtgcatttactgcaccgccctgtgcagcggaccaaagggactgacagaatgatctgctagccactgcgtgttcaggcagggctttggaactgccctctgctaccaggagtggtccaggggctttgatacatcctctgctagggcctcagcgtacatcgaaagcagaggaaaagtaagactcccttgactttcaacttaagtcccttcaatgacactcctttctccccagccagccacccaggctggtcatccagggatcagcccgggagcctgggtctccgcagtacctggatccgcctgcgccgaaggggcagctcggtgctccgcggcgctgttgaatcacggttgagtcacccctttcggggtctcggagtttcctacaggaggggagtgggcggggcaggggtgtggactgatcgggagccgcccaccgcgaaagcgagtgacccgggccggggggcggggagtcgcgagcttcggcggaggcggcgcggggtggagaggagccggtgtccgcgcggccctttataacgcaccccgcgccgaccccggcagcctgggtacagccggtacccatcactcgctccctccgatcgctcctgtagcgtagccaacgcgccgccgctgaagccccgagacctcgcctccagcccttggacatggcgatcctcgtgcgcccgcgcctcctagcggctctcgcgcccacgtttctcggctgcctcctcctccaggtgaccgcgggtgcaggtgagtggcattggtcctgagaagccgggggcgctccgcccgcgggaaacagagaacctatctggctagctggggggggagggggggtgctgctctggttccttggggacaccaaggtgtccggctgtacctgtggatttggctgcgctcgccctgaggatttgaatgaacaatgggatggaaggtagttggtgcccggactcctggcagttgtcactgctaaagatgcctgctttgtgcgtccaccgcggttgcataagataaaactgagattccagctttaactctgagggagagtttcctagatgttgggcccttgaaggtggcaggctcgtgggcccgcctttgccaaagtcggcgtgctttgcgctttgcgtggcttgcatgttatctctgacaaggtgtgcgccttcccgtaataaataggtgttcttgtggcctttggtttcggtctgatagaagcaaggctgttgtaaactcaggcaagacagagtgcatacccacatctacccagtgctgagttgaggcagaggctgaaaccagggggcttgcaaaacgagacgttgatgacagcactttaatggaatagcatcccaccatgtgacaggtgccacgtgggctgcacgtctgacgagctctccttgagaatttcagtgtaaagatattttagattacaatgggaaaagtccggttagactcttttttttttttttcgtttatgtgcattcctgactctcacgtgtgtgggggactagccctgtttggaatttattcaaacccttccttgcggcctctccgacggcttcggaagagtccgggactctctggcaccttgggccctccttctgtgtcgttttaaagtgggctttccctggaatagtttaaaggaggacttcaggatcttcagctctttccacggattgcagtatgggtgttgcctagaggactatatggccctgagggcggggatggggcgtggggggaaggacacgtacacacacacacacacacacacacacacaccctctgctcttgacaagttagcaacgttatttttgtgactggcaacaaaacatgtgctccctttgatggcagaggctgggaaagtgtttaagacacaaggtcgagaccacagaactgggtgcaattcgagatattttgtgcttatttgtaggcattccagagaaagcgtttaatttaacttggatatcaactgatttcaagacaattttggagtggcaacccaaacccaccaactatacctacactgtacagataaggtaagccaggttaaaaccaaagcaagatggctaacagctttgcacgtctagctcttatgcactaatagggactctttgaagtcagttgttggatggggctgtttaccaaatggtagagttgtcatggtaatctgctggctactgtgatgttccatcaaatggaaacattccatatgctaaactgatatgacaggaactggcgcgggggggggggggttgtacctctgtcccttgagtgcttaccaagcacacctaagtcctggtttagatactcaacccagcatagaattgtcactgtggtcactgtgttgctcggggaggaagtagaagtagaaattcagtcactcttagctgtacatagggagttccaggccatcctacaatacgtgggaccctgtatctaaataatgcagatacatacatacatacatacatacatacatacatacatacatgacatacatgacatagtgtctggttctttgagggtagttataagaaaattaataagtgcatataagctatagaggggtgagcccacttacacaggcacatagtgtgctttgggatcgtccgcagctagtctcctctaagcaccaatgcccttttctggttactagctgtctagactgagctgctgcaggcatgtcagggagaggtaggaggaattccagtgtctacactgccctgaagcagtgcttgattcattaatctatggactcgttgaattgaaatttggttatacacacacgatttcggtggaggccaaattcctgagtcattgagctgcttagaaaacactgaaggatttgtctgcatcctgcgccgtctggggctgttcggctcactcagccctgttcaccgactgacagctctcgggaacaagtaatcccgtgcttttcctctaagtggtaaccagggtgtgccaggtgccttccttgatgattttcacatatttttggcaagcttttggtttctgatagattctaggagatggataaggaacttaagaaacctgcctggtgtggaaattgttttaatagtgacgatgtaaaggacacaggagtatcgtaggcaaaaggcaggcacattgtaaccctttgtttttccattttagcttatgttaagccttatttagcactaggatgcaactctcaggtgtggatcctttaaatgactttcttgtctgatagatattgccaaaccaaaaggggagagaatttgtgtgtgtgtgtgtgtgtgttcatgcatgcatatatgcacgtatagctatgcatagctaaatagagatgaatcttataatctataattaccatcacagtcaaacttatcagttcaaaggctggtgatgcaggtcagttgtagagttcttgtctatcgtgtacaaagccttgggtcccatccattacccatgtccactctgctcccaatcttgttgaactggtagtgatggcacgctgtaatccctgcacagcttgctcatatgacgcaggaggattaggagttcaaggctacatgtttgttcaagaccaactggagctatagagtgaaactctgacttaaaacaagttcaaaccctgacagttgagagtcattaaagtagttaatttctcttatatagatctaacatttaccttaccctttccttctagatatgaagattaggtttttcagagaaatggttactttctaatatcttgaattaattcgaacaacattgggaatatatcttgggttgtcttgattttttaagatagaagtctgtattggttgaaagttttggagtttttaaggtggtcaccattgaggtgagcacggctcatgatctgcagacatcagagctgaggacatagcttggggatccctgcagacttgggtgtctcactgctcaggtcaccttgctcaggtccttgctgctgagctgggtgatcctggaagcattgccattagtcaaggcatgagacccgagctagccttcacccttcccaggggaaaggggattccaggtctaaggggatctggaaagccaggggttcacactgactgctggtggtacctgcccaggctacctggaatggaggtgtgccacgtctgcagggggctcttccagtctgctgggaggaagggggaagctctgctcatcctgtctcctgaatctcctttattcacctccctgaggaggtggcaccttatctaggggaccctgtcccccacacccacccatgctgctggagcttggctgtgtgatgctggccaaagggcagctctgagggacccaagccccttggctgagtgtggacggtgtcttctctgaaagcaggctcagtggtaattgggaagcaacagcggcccagccttagcaccgtgacatggctcgtgctgtccatccagtccatagtgtgtgttgaattccaaagctgaatttagaagcttccagacacagcctctaactcaatgagtgaatgtttcttttctgttggaactcctcagtaaattgggggggggcagaatttatttccaaatttggggagtttttccgctaacctagagggaccttgaagacttttaaagtggtttgtgctcttagcagcacctggaggacctcaatggaactgtctcctcattgacctgttatcacctacattttatttagagagcacatggtcctacttgtaagctgggtcaaacaaaacactgcatccatcaggcgtatgacttcacccagagcccagccactgaacgacttgcttgtcatatggtggaaagaaaccattaaggtgtcgttggcttttggtatagcaaagatgtaatcaaggtacattttggtcattctggatccaaactgcctctctatacttattatagaagaaagttcaaaaatacaagaaactaaaatatttcaattttaatattagctttcgactttgaggataagggatatgtgtacctatgtactcgggttctttcaaagggctttagagaattctcatttgtgcctcttctgtgtggatttagataattttatctgtatgtgactcccctgctgaatgttttatcatattacacatctatgttgaataataaggtctatcaagccgggtggtggtggcacacgcctttaatcccagcacttgggaggcagaggcaggcgaatttctgagttcgaggccagcctggtctacaaagtgagttccaggacagccagggctatacagagaaaccctgtcttgaaaaaaatcaaaataaataaataagtaaataaataaagtctaacaataatagattccctacataattatattccgttacgatcatcatatatggctagtcactgactctcctatatgccagatattctgctgggctctttgaacgtgttagctcatttgctactatctaaggaggtgctgggttctttttaatgtgtgtgatcatgggaagtgtgtattcaccccgaagggctttcacactcggcaaacacagaagccctgatgcttggccgagagatgagaaccagcactcttgggccaaagagatcctgcatgtggagagctttcggggcagtaaggttgggggtggtcgtggtgaccagcaagcaggagaagagcgctaagcagaatgacatggagatctggaactcgcttgagtgccgaggtctgtctagcaagatattaggacctcagagcactgctggcattcacctgatggagtgcatctggcaacaagtagtgatgcttggctaatggtcattgtcctctcaaagaactttattgtcagcagaagtttttcaagttatttctcttcgatggtgccttacaaaagaaagatctgccctttgccctcggaggctcagactttgttaagcaggcattatctgtattgtttctctctgatctttggtgagatgtgtttctcattctcgtaggtaaggctagcatgtggtgtaacgaagcaaacaaacccagctctaccacgcgtgttagcgtgtgactgtgaaggctgcttagtgccactggccttccatcctcatgtacatcaaaaataaaataaaataaaataaaataaaataaaataaaacaaaaccaaataaaaggtgcccagcagagctaacctggttactgggtgaacttgcatttgtacttcagatgctcttcaggttttcccagttgggagcgcagagcactccggtggaaggccatccatcctgctagccacagaccctaacctcccgtttcttttcctccttcctccctttagtgatcgatctagaaactggaaaaacaagtgcttctcgaccacagacaccgagtgcgacctcacagacgagatcgtgaaggatgtgacctgggcctatgaagcaaaggtcctctctgtcccacggaggaactcagttcatggagacggagaccaacttgtgattcatggggaggagccgccatttacaaacgccccaaagtttttaccttaccgagacagtaagtagcttggtttgtaatgccttcactcttggtctaggcttctgtataaatatgcttgtgcttcatgaccccctattagccattaattagatctctctctctctctctctctctctctctctctctctctctctctctcacacacacacacacacacacacacacacacacacacacacaccaaggagatgcatcttataagtgtctctctctctgcagatgggcaggaggttcacagaatgtgaactcgtgtcttgatcactatgtcatctactcactttctcctccagtctgccgccacttcctatcaggaaccagctggagggtgcgctaaagctggggttgtggcttttcctcaggctactgtgtcatatgctgaattttatattctttcatcagaccaggggtgctagtttagacattttcctccaatgagcacagatccacagggaagccacttgtgcctgaaattgggcttgtcagcctccctcgtgctgggtttgaatgtgtagcatgctttcccacctttgtccaagtgtgtgagcctgccatctaacaaggaaatcttctttcctttctctctctctctctctctctctctctctctgtgtgtgtgtgtgtgtgtgtatgtatgtatatcttccttcctttcttttttctttccttccatgtttcagaagtagacatttcacaagcttatgcagcacattaggaagactaaatcccacttcttttgcaaggcctgctggttaaaacacaagtgttgtggttttggtaatccccatctgtccccttctgattgctcagcgacgtggatggtgagaaggatgaagggcaattgagaagattttacttccgatgtttgctctaatggtctaactacccaaatgaatgtagctgccttcctggggtccccactgacaagcctctcttcggactcctttgtatttttacctttgtgcttctgcacaacacgcgcatgccaatcatgctccccaagctgtgatttcccttcatagttcttgagctttcagcagtccagaccaaatcctttgagatacacaacagaacacacacaatcacacacacacacacacactgttctgtgatttgcacagagtgaggacttgtctgaccatcacatgtgctgcagttacctcgtgtaactgaatcaggaaccaagaacttaaaatagattgggtgtgggatgtgtcaagaggttgttgcaggaaaacattaaaaattaacaaaattagaaggtgacaaatgtgcacatagtggactttcaaaaaaaaaagtctatgcctggtgtatgtttgtgtatcatctgtaactcagttccaatgatcagatgccatctgacctgtggaccctagccatgtatgtagtacacacatccatagaacaggcaaaatacatgcataaaaataaaaaaatatttttaaaagtcttaaaaaaaaaaaaaaacccttcctgggcttcccagcctctaatgcattgtagctaaaatacggaagcttggaaaacttcttgtccagcctggtagccatcactcacacatggctaccaaacttttgaaatatggctaatgttgccaaggtgaaaaatatatgccagtttttgaaaaatataaaatatccataatatccactatattgagtgcatgttaaaatgccaatctgttaatggacctactaaagtatataattaaagttaattccacaggctccttctcacatttttctaacatctgcaaggaagggtctccttgtgcttccagaggacggcatggcttgctatgtgttgagactcagctacaagaccgtcaccactcctgctactggagctgattctgtacaggtgttctgtatgagcgatggaggagctgtcaattacgataactaagtgatcttttctttgtattgatttttaatttctaagcaaggtatttttttttccatttcagcaaaccttggacagccagtaattcagcagtttgaacaagatggtagaaaactgaacgtggttgtaaaagactcacttacattagtcagaaagaatggtacattcctcaccctgcggcaagtctttggcaaggacttgggttatataattacttatcggaaaggctcaagcacgggaaaggtaagagattttccatttattttatgccccccccctttttaatagatctttgattctccacagcatgcttgcttcccagttcaaatatggaggaatgggggttgacatggtggtgtgcataacaggtcctccttctgcctgtcactgctgaaccttctgcccttgggcagggtcctttcagagattccagtcacacagtcaaggagctagaccagactgtcctctcccacatcttcatattggggaccccagggtgggggtggagcagggataacttattaactaatttgcatttgtgtgcacatgtgtatgaatgcatgtacaggcatgtgtacatgtgcatgagagtttgtatgtgcatgtgtgtgcgcatgtgcatgtatgtgcttgcgtgtgtgatgctgggcagtgttacccaggaccttgtacactagacaagttctctaccactgagttgcattcccagcccccaaatgccccttctttggtgattccatctctcttttcctacagtaaaaaggggctgaactgggctaggtgtactgtctttttagcagtcatcaggggttaccgatgtgccaagtgacttctaatccctcgtgcgtcaatgatgtctgcatggacttagagccctggttttgcttttctcggtgatttgagaactctatcttttcacagaaaacaaacattacaaacaccaatgaattctcgattgatgtggaagaaggagtaagctactgcttttttgtacaagctatgattttctccaggaaaactaaccaaaatagcccaggaagcagtacagtgtgcaccgagcaatggaagagtttcctgggaggtgagtggccatggctgtcagtgccctgggaggctcggctgcccccacatcaaagccaacatgtatctcttagtttcaaacattcagaaatgagcttagggtgtcttagaatgtctttttggattcttcattctctttgggggcatttaggaatgatttcacattgttcccttggtcacttccctgggcacttgcaatgggccaggttctatagggtactgagcatgcagattacagactttagaacttaggtaccaagtcacagccttctctgcctgtttttcctactgacatgatctgaggggcctgggcccaggggaggagtagtttctggaccagctagtttctggaatggcagatatttcagtgcacctatgatggactgttggtacaggagctgtgtgtgtgtgtgtgtgtgtgtgtgtgtgtgtgtgtgtgtgtgcatgaatacactctctgggtattcagtgttgtgactaaaagctctgggccccacagagaaggggctctgctgtcagaggagacgactaagggggcagtagctatggcctggctctgttagagcttcaagtcattatgccaactaggccagactcgggaagacaaatgccacatcttcctcttatatgtggaaagctagattaaatgtatgtacatgtatgtatttatatatgtatgtagtggggtcatgaaactagaaaggagaccatgggagagaaggaagaaatgtaaaggggaagggttaataaaatagaagtactgaaaagaggcgggggatggaggaactatctagggaaagaaaccagaaagagaccaaggggcatgggggccaggaatgaatgagaacaaagtctaattgcatgttcttcataaaaaataccataattaaacacattactttgtatgctggcctaaaaataacttttttaaatgcaataatatgtttaagtattctgccttcttgcctctgttaacagaaagttctctttccattggagcccattcccactctgggctagccagctccttagccacaagcctcactattcccttccctaagccctacactatctagttctgatgccccaggctctgcctgtttccctgcttcatctcttaacccaatagatcctctggggtactgactaccccagccaactactgtctcttcgtctccaccatcagcactgtgccccctccttctctgtctttcccatttatcttctgttataatctctgccaccaaactcagggtcgttttttggactacatttccaacatgtgttcttccatccatctcccgtggctcctttctagtccaggaaccttgggtagctcatttaatgctgtagccactgtcagtttctctcaagcgtatgaggtcagataaattcttaatcaaagaatgagagaaagagtggaagaaataaatgtcgggtttcaaaaaggcgggacggtatgatttggggtcacgcccaccggcaagtggcgttcgagcctccatgttgactttacatctttgatttatttttttgtttgtttgtttgtctgtttttgcttttcagaaacactcatcattgtgggagcagtggtgctcctggccaccatctttatcatcctcctgtccatatctctgtgcaagcgcagaaagaaccgagcgggacagaaagggaagaacaccccgtcgcgcttggcatagaggaaaggctgaagccgctaacgctcacactgcctgcacggcactgttgcggagagctctgatgggaactgtgcaacatggagcgtggagcctgccgatcctagctcagagaggctgccttcatggcctgttactccagctaacgctttgattccaacactagcatttgtcacgttaggacgaactgaaacggtacaaactggttaacactacagcgccttttgcacaaatgctttagattgtatggtctacactcaggaagacactaggtcacccaggcaaagccagtggacagatgcctttcatataacctgggtgggcttttggaaaatctttgagaagttgatttcataggctgtagaacagtaaagtgggaactgggcggacttttctaacagtcgtacttttataaagcggtatttgggtgttttttccctcgaataggtacttttggaagttcaaagcaagtggcaaactttcatataaacatgttaaatgcaggatatttctgcttggggcatctttgtgatttgtactttcctacaatttagcactttaactgacaatgatggggtttaaacatttgacagccaactctatttttatacgactactatacaaagaaactacatatagttttatgatttaaggtacttagaaatgtttatggttaacattgtatatatttacataaaatttaaaggttttgtatatgggattttctatttatatagcttctatttgtatattttgagataatttatttaatatacttttatataaataaaggtgattgggaattgagacaatcgcatttatatcttccacttattatttacttcttggttttcacattaggtgactccgttgctgtaacaattgaccttatcagtcttagtggcttacaacaatatatacttttcgcttagactacgtgtttgtgagtgttggtaggtggtagctcctaatggtgtcttctcattacaaggcccgaaacaaagcaggggtctctattggatcagagcaaaggtgaacacacacacacacacatgcacgcgcgcacacacacacacacacacacacacacacacacacacatacatgcgtacacccccccccccccaaagcacaaccataccaaggcaaacaaaacaaaacagcaaaacaggctgctacaccttcctttttttttagtttttcgagacagggtttctctctgtatagccctggctgtcctagaactctctctgtagaccaggctggcctcgaactcagaagtccgcctgcctctgcctctccagtgctgggattaaaggcgtgtgccaccacgcccggctacaccttccatgtttaagccactttttaaaaatgactttttgattataattacatcacttccccccttccctttcctccctgcagtccctccctcgtaatccctgccctttgacccttagactcattcgaattcatagtcttaacacttccaccgaatactttggttcaccagtcaagcaagtcacgtgggtaagcctgataaacctagcctactgagagagaccatctagtacagacttagggttctccccagtcactcacttgcattttggaggaggctcaaagaaaaagagacctgccttagattttgttgttgttgttgataaaaattggaaggatcaagttagacctcatgtgaggaaactaagcacaaggactcggtcatgctgagttgactcagtgacttctaaaacggagaaacggagcgagtccttcagaggcagcaaaagtctacactgcgcagaaagaagatctgaactgaatgatggtgttctgagctggctgccaggcaaacctgctcttgcttccctccctctgtgcaaggctggggagttgccagtggccctcttaactggtcggttttgtttactcaataacaaatgcgctccatctgccccgctgcgggcaccagtgcagcttttgataccacagcgaccagtgtgtgtggtggagtcccaaagagaaaggacaagcatgtgtccttgtcctcagagaggggccttacgccttatcttgagtccctgctcacatgacttatccaggacaaagagctcggtgtagcacacgaagggcaaagttggctacaggagctcatgactgaccttgccttcctctgccaggaataattatagcattatgtaatcccctagcttcataatctaaggaatttgaattatctgcagactgaaagcaggtctggatgtaacaaggtagagcaggaggaagtcgttatagatgcggtgtagcggtgctgcgggaatcagaaggttccactcccagcgttaaccattgcacacaggccccctctctccttgttgtggacatgagacagctgctcctaaatcaacacaacgggttctgcagccgaactgctgccctctagtgggccgaactgaacgtctccctccctgtgctggagcctatcaagcatactatattaattactaaacagaactcgaaagctctctacagatacattttccatcttaaattactgtttggcatatatgtaagagactctttttgaactctgagtcagagcaactctctcttaaacaatcatcttgctcccttagggaaaactcatttctgaatgcccttcctatgagctcctggatttaacacgtactctttaacacattctcttcccccaactgttgcctcgctgtttctttttttctttattataatcttggctttaatagagtgggatcaatcctatttaaggagaggctcccgcaaatgaccagtatgcacatttgtttaacatagcaggagccctcacttaatcacacacaaaaggatgctctgactgggtgctgcacacagatgatctgtagagcaaaatgagaagacaagttggggacgtgacagactcctggaatacacttcacccaaagcccccaccactggaaacccaactaattattttagcaagctggagctgggttttctgttccctgtgatcaagccttctgtcctttgtagttaaaaagaaatacccaaactaacattgtattatgaaagtcaagcataaagaaagaagccagctatggtggtttgaatgagaacggcccccataggctcatatgttcaagtgcatggttcccagctggtggcattatttgggaaagattggccatggtgggtgtggccttgttggagtttgttgctgctggtggactttgaggctgaaaagtccattccattcctctctctctctctctctctctctctctctctctctctctctctctctctctccccccatcccaaccgtcaaagataccaagaactaaaccaccaacccaagagtacacacgcagggcccca

SEQ ID NO: 4 - DNA, 21880 bp, Artificial Sequence
  unsure (3465)(3466)(3467)(3468)(3469)(3470)(3471)(3472)(3473)3474),(3475)(3476)(3477)(3478)(3479)(3480)(3481)(3482): Humanized tissue factor allele sequence, using; mouse tissue factor gene from the ATG; initiation codon in exon 1 through exon 2 from; sequence No. 3 is removed and replaced with the; open reading frame of human tissue factor cDNA and where n can
  equal a, c, t, or g.
ctaccctgtcccatgaggccagagagaaggctaagaccaggtaagactgttgttccaagggttcagttcccagcactcccattgggtggctcacaaccatctgtatctgcaccttcagagactccaaccccttgtctcttcaggtccccacacacatgtcagacaggttcactctctccctagcctctaacacacacacacacacacacacacacacacacacacacacacacacacacacacagagggaggataaaaagaaatcctttaacttgtcccagaggcaggctctgaggtcaccactgggaagagcacactaagtttatggaagtgagtggcttggggagccaggtcagtgctggggccactggggcatctcacaggagtaagcttcttgggggagactccctctggagctctccgggagggagggaccgctgaacagacagagccaagcaagtgcaggtgattccatcacaagtcccatcttgagccagggtggtggtgtcatgatgataaacagcagacaggcaagaaatgtgctgcggaatggcaggatgataagggggaactgattgcaatcaagcacactcatcagtttccttagcaagtctgcaaggcctttcctaccccaggttgtgagttggaagggtgacccatatttctcgatgaatgacagaccctgctgcgggacacggatgccatgttcttgaggcaaataagccattttcagttacaatatctttcttcttctctgttctgggtaggagtgcaacttgaatgtagaaacaccagaacaaaataaactggaaacaaggcaaaagaaagagccctttcttctcaactaagaccagatttggcttggttttcttttggaaaacctccatattctgaagccaggcactctagagaggcacagctcacaaggtagggaacactgagggcagcagggacagggacttgaattcatgctgcaatttcttttaaaggatttatttgtttgtttatttatttatttacttatttattttatgtgagtacacacactgtcactttcttcaaacacaccagaagaaagcaccaggtcccattaaagatggttgtgagccaccacgtggttgctgggaattgaactcaggacctctggaagagcagtcagtgctcttaaccgctgatgcttctctccagcccccatgctgcaatttccacagtgctcccatgggaagtgtgtgttaccgcacattcagtgagacaggaagcactgtaagacagtaagacatagcactgcgattatgaagttgtactcaagggctccgatagtcagatgcaaccccagctcaccattatccagttgtgtgacttctgaacaacttgttcaactgcctttggcttcagttaacccatctgtaaattcatccctatctgtcaaaaatgggattactctgaggactaagtgacttaatacttggtggctaataaatggtattttctggctattttcaatgtcaggaagattatagcatggtcctgagattcactgagtttccaaagtttatctgcttttccactgggacatgatgtcagctaagaagaaatacaagagggaataaagttatttatagagttcctggattcttagaagatcttgcccatgacagaaaccatcgtaatcgtaattgtcatctgagccgcatttcattattgcctaaggccagctggtagcatgtgggaatttgggtgttgtatggtaatctcctttgaactaaaaaattccatgctggagagaagctcatgaatgctcaggaagtatttgtgtgtgtacatacatgtgtgcatgtttgtgtggatgtgtttgctacatgcagacaacgtcagtagaggccacaaaataagctgcctggtgtgggtgctaggaactgcaaatctctgtaagagcaacacgtgcccttaactgttgagccatctttccagtcccaacacttatagaaagttttaaaggtaggttatttaagattgaggaaataaacaactcagaaatctcagaaactcccccaaatttatcaaattaacaaggctcctcccttcccatggctttacatgcagtaagaactgctgaggggaggagactttctcacctgtcaagctggctacaggtcatacagagagctccaaggtcccagcttttgtgagttttcatttatgctgggtgggctttggtgatacaactgcctttaaatcattcctccttctacgagaactctttcatccatatttctctaagtaaccccataaaaactcatttgtttaccgagttggactttagtggcattgttactttgatctgttaccagttccctatctgaggtgagtaggcttttgttcgtatctcccccagaatggtttcacataactcggagtttctaggctttagagcagggataaaggcaaattcctagacaactgagtcttaggtcagagcgagctagactgaaaccatgagtaaccttgatgatagagaaatcaaagtttggggaagttgatctgaatcactgatatgcatctctcaagtcagcagtagtttcctttcttgctttaacccaaagaagtgggaaacgtaacaagatccatgtgtcttttaggacctgttgtaaccacacatataactggattttaccaagtcaagtttcatggtcttctctttctgcaaatttcaatcctcgttccttctccagatcctcccaggtcacctcccgccctcctctagccacacaccaaggaccagcatcaagtcttgttcctgtccgttatggcttggatgaaaaatgtcccccacaggctggtgtatgaacatttggcatctcatccgtggtgctgttcagggtggatgtagggttatagaacacttaagaactagagctttgctgggggaagtgcatcactgaggagaggctttgagtatgtgtagcctcacccaacttccggttccctctgtctgcttcctgtgtgtagatgaaatgtgacctctctgattcctaacagccagttcgccgcctgttccggcttctgtgtcttccccagcgcaatggaccctatccctctggaactagaagcaaatacaaatcccttcttctttaagttgattttgtcaggttattttatcacaacctcaggaaagtaattgacacgccatctgtccagtagctttagcagtcctcttctctggtctcagaccccctctgcctccaaaataggtcacacagcccagacttggatgttcctctggcaactgagattgcccacggatggtcccagatcggagcagaaacaactggaatcttatagtagtactggtgattggctccagatgtgacaaagccaagacatgtattctgggctgttggtgacttctaggcactgttccatttgttctaagagcttcctgggctcgtaaactgtaactttttnnnnnnnnnnnnnnnnnnccacctcctccactcactgatctcagcccttgccctggcctttgctcttcctctgtgctccagtttaccatgatctgaatgttggtkycayctgaaaatgcatatgttgaaacctcwtgcaggatacaatagtatcaagaggtgggaggggtttctggatgtgattaagttaaaaggctccgccctcataagttatatagaggatcaatgcccttatgagaaggccatggtgggtcactccctctcccctgctgtgaggacacaggtcaccatctttgaagcccagaatgtgcctttgagaaaccctgattctattgacacttttgtcttagacttctcaggctccagcactggggtctttgttactgcaaactctcctgccttattttcctccttcctcctctcctcccttctttttcccctgccctactttccttcttgtgtatagctgccaaagtgatcactgaagtattttgtaagagtggcctgaatggacaaagacgttgctcaaactctgctttcccccaaggaaccttctctgatgaactggtcagaagaaagttctccagggggtcccgagtagttttacgttataaacaaagctttaaggatatgagaacctgtcctgggaaacctgtgtattaccaaaaccagagcagtcactattagcatgaagttttcagtctatgagtaagacatgtatctcagacctcagttctgctccatccttgtaggaagtgtctatttgtttttctccctgacttataagctatttagagagagagagagagactcaaacatagttcctgtggtatattgtcccatacatagtaatccctcaataaatatttattaagttacacaaaccaagatcttcattattccctggtgataggaaagacaaacacttaactttttctctttttttttctttctttttttttctccctttccttggttggttggtttggttttttgagacagggtttctctgtgtagccctggctgtcttggaactttctttatagaccataccaggacagcctcgaacttagagtgctgatcgctaagataggcagcctactagccctgagtctgcacaggtacttatgtgctaaaaatcccaagacttagactggaagattgtcctctaaaatcatggggctttagattttcttcttaaagataggtgcagtggtgcatgcctttaatcccagcactcgggaggtaaaggcaggtggatctctctgagctggacacagcctggtctatactagtttcagtccagccagggagacataatgagaacttgtctcaaaacttaataataagtgaataagcagatagaaacagtgtttttcaaaatcttggtgctatgtgataactaagttagtggcctagcaactgtattttgaatacagtttttgaatggtcaggttaacgttatacaagttttcacctaattttggatgaaatataaactgttggatagtacataattattttcacaaaactcctgaagcacagcagccattttctccatgactaaccctgccagatgtttcttcggttttgctgagtcactttttctcccgactcagaactctgcgttctcactaactctgaagttccactggcattttctcatattttgccttttacctgaaaaaaaatgggaaggggggaatggttaaattgcttattttactatctcacacgcatataaccagttttttaaaaaaaaaataattactttcagttttaccttcggtcataaatcttaacctgtccttttcctgggaagacagacattttgaggaatttatgctaaacagcctttaagcctggctatcaacagtcacgttgcatcccatgactgagcagcttctgaatcaccctcgagtgtaagctgttagaaaaaaatgaaagcagcccccaaacgtcttcattctgttctgaaactacctcttctccaggtagaagtcctagaaaccggaaggaccctcagccaccttgagcgggctggggctggctggggagcaaagtccttggaatccataggtttgcatttcctcaccaatctgatttgctcactgcctggctcccagacagaagcgcttcagcgacaagagttcatctggtgaatgttaatgaaaactctcagcgacaagtgtggctgcataaatgaattttattgattcaaatgtgtcaagttcctcctgggtgcttgtaaactgtgctaagagctaaaggggggacctgatcccccctccccctaaaagagcttgatgggggaagttgacagagggcaccattgagattatgtaaagtacttgatgaacttggttggtaaagctgttagcatgctctataaacgtgcacaagaagggctaaaagcgtgccctggtcacttacagactcctcatcagccacattgcaccactggtgatgcaaaaggaaactgggctctctacgtgtcttcacgcatgtctgctttaaggaatagccaacaggatatttctgcctatgaacaataagttccccaaaccgcgagtgtttcccacacctgctgaggaacacagttgagggtccgtggatggaaaaaagatcaccctttgaacagttttcttgatgccctcttcctttgctcagttgggagtaaaagaaagtgcggtcaagagggaaagtccctggagtagaggaccagatagagctacattgtgtacttaggccagtgagcacacggaccagctgacctcaagattggtattgtagaaggctatctaagaggttctgccctgtccattctcagacataagcaggaagcctaatcaaaagctactgtgagtgggacgacagcttagttactctttacacttctcagagcctacaaggtggacccaagtttgactttactcaaggaaagtaaaaaaggaaggaagaaaggaaggaaggaaggaaggaaggaaggaaggaaggaagattgtacaaagcatccaaagacacagatatttacattacagttcataaccgtagcaaaattacagttatgaagtagcaataaaaataattttatggttgggatcactacaatatgaggagctgtgttaaagggtcgcagaattagggaaattggaaaagaagggataaactggacagtcaaccaggagtgacaagaaatcgtttgatacactgtcgagcaaatgctaccagtaggataagtgatcgtctaaggcttcaacaacgtcacggcaaagcctagaacaggaggccaggaaataaatgaagaaaacccaaagcttgcaagtgacacctggctgccactaggctcagtgacagaggtgggctcctgggtccattcccagcatccaggaagggaaaacacaaattggaacgtccgaatgtttatttttcagtttcattttttgtttttctgcgctttggaggaattcactcaaacccactgcgggcttcctcaacgtgagaatcatccttcctggaacccccgtggctacagaagtcacgctcaaattctctcaggcttcatgttgcagttcctcggtcacctcgggaacgggagtggggtgccaagtggcctttagacctaatccagggcccttgtccatggcagacacgtgcatttactgcaccgccctgtgcagcggaccaaagggactgacagaatgatctgctagccactgcgtgttcaggcagggctttggaactgccctctgctaccaggagtggtccaggggctttgatacatcctctgctagggcctcagcgtacatcgaaagcagaggaaaagtaagactcccttgactttcaacttaagtcccttcaatgacactcctttctccccagccagccacccaggctggtcatccagggatcagcccgggagcctgggtctccgcagtacctggatccgcctgcgccgaaggggcagctcggtgctccgcggcgctgttgaatcacggttgagtcacccctttcggggtctcggagtttcctacaggaggggagtgggcggggcaggggtgtggactgatcgggagccgcccaccgcgaaagcgagtgacccgggccggggggcggggagtcgcgagcttcggcggaggcggcgcggggtggagaggagccggtgtccgcgcggccctttataacgcaccccgcgccgaccccggcagcctgggtacagccggtacccatcactcgctccctccgatcgctcctgtagcgtagccaacgcgccgccgctgaagccccgagacctcgcctccagcccttggacggccgctctagaggccatagcggccatttggtggcggccggccgctcgaggccaccatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaggatccgcagatcctgctcctgcgctggcgcctgcaaatgcaaacaatgcaaatgtacttcctgcaagaaaagtaagttggatcttctctgccatttccccgtcactctcctggggtccctagcccgccgcgccgcgccttccctcccgggagcgttcaggtggtgtgcctctgacaaggtttctcgctcacgttcaactcttctctccccacaggctgctgctcctgctgccccgtgggctgtgcgaagtgctcccagggctgcatctgcaaagaggcttccgacaagtgcagctgctgtgcctgaaggggggcggaggggtccccacatctgtgtaaatagaccatgtagaagcctagccttttttgtacaaccctgactcgttctccacaactttttctataaagcatgtaactgacaataaaagccgttgacttgattaattcagcttgtctctgtgcattggttaagggctggcagaggcgttgagatgggactgcaggaattcgattggccggcgcgcctaatttaaatataacttcgtatagcatacattatacgaagttatggcgcgcctcgaaaatggccagcgaggccggtacccaattcgccctataggttaaaaccaaagcaagatggctaacagctttgcacgtctagctcttatgcactaatagggactctttgaagtcagttgttggatggggctgtttaccaaatggtagagttgtcatggtaatctgctggctactgtgatgttccatcaaatggaaacattccatatgctaaactgatatgacaggaactggcgcgggggggggggggttgtacctctgtcccttgagtgcttaccaagcacacctaagtcctggtttagatactcaacccagcatagaattgtcactgtggtcactgtgttgctcggggaggaagtagaagtagaaattcagtcactcttagctgtacatagggagttccaggccatcctacaatacgtgggaccctgtatctaaataatgcagatacatacatacatacatacatacatacatacatacatacatgacatacatgacatagtgtctggttctttgagggtagttataagaaaattaataagtgcatataagctatagaggggtgagcccacttacacaggcacatagtgtgctttgggatcgtccgcagctagtctcctctaagcaccaatgcccttttctggttactagctgtctagactgagctgctgcaggcatgtcagggagaggtaggaggaattccagtgtctacactgccctgaagcagtgcttgattcattaatctatggactcgttgaattgaaatttggttatacacacacgatttcggtggaggccaaattcctgagtcattgagctgcttagaaaacactgaaggatttgtctgcatcctgcgccgtctggggctgttcggctcactcagccctgttcaccgactgacagctctcgggaacaagtaatcccgtgcttttcctctaagtggtaaccagggtgtgccaggtgccttccttgatgattttcacatatttttggcaagcttttggtttctgatagattctaggagatggataaggaacttaagaaacctgcctggtgtggaaattgttttaatagtgacgatgtaaaggacacaggagtatcgtaggcaaaaggcaggcacattgtaaccctttgtttttccattttagcttatgttaagccttatttagcactaggatgcaactctcaggtgtggatcctttaaatgactttcttgtctgatagatattgccaaaccaaaaggggagagaatttgtgtgtgtgtgtgtgtgtgttcatgcatgcatatatgcacgtatagctatgcatagctaaatagagatgaatcttataatctataattaccatcacagtcaaacttatcagttcaaaggctggtgatgcaggtcagttgtagagttcttgtctatcgtgtacaaagccttgggtcccatccattacccatgtccactctgctcccaatcttgttgaactggtagtgatggcacgctgtaatccctgcacagcttgctcatatgacgcaggaggattaggagttcaaggctacatgtttgttcaagaccaactggagctatagagtgaaactctgacttaaaacaagttcaaaccctgacagttgagagtcattaaagtagttaatttctcttatatagatctaacatttaccttaccctttccttctagatatgaagattaggtttttcagagaaatggttactttctaatatcttgaattaattcgaacaacattgggaatatatcttgggttgtcttgattttttaagatagaagtctgtattggttgaaagttttggagtttttaaggtggtcaccattgaggtgagcacggctcatgatctgcagacatcagagctgaggacatagcttggggatccctgcagacttgggtgtctcactgctcaggtcaccttgctcaggtccttgctgctgagctgggtgatcctggaagcattgccattagtcaaggcatgagacccgagctagccttcacccttcccaggggaaaggggattccaggtctaaggggatctggaaagccaggggttcacactgactgctggtggtacctgcccaggctacctggaatggaggtgtgccacgtctgcagggggctcttccagtctgctgggaggaagggggaagctctgctcatcctgtctcctgaatctcctttattcacctccctgaggaggtggcaccttatctaggggaccctgtcccccacacccacccatgctgctggagcttggctgtgtgatgctggccaaagggcagctctgagggacccaagccccttggctgagtgtggacggtgtcttctctgaaagcaggctcagtggtaattgggaagcaacagcggcccagccttagcaccgtgacatggctcgtgctgtccatccagtccatagtgtgtgttgaattccaaagctgaatttagaagcttccagacacagcctctaactcaatgagtgaatgtttcttttctgttggaactcctcagtaaattgggggggggcagaatttatttccaaatttggggagtttttccgctaacctagagggaccttgaagacttttaaagtggtttgtgctcttagcagcacctggaggacctcaatggaactgtctcctcattgacctgttatcacctacattttatttagagagcacatggtcctacttgtaagctgggtcaaacaaaacactgcatccatcaggcgtatgacttcacccagagcccagccactgaacgacttgcttgtcatatggtggaaagaaaccattaaggtgtcgttggcttttggtatagcaaagatgtaatcaaggtacattttggtcattctggatccaaactgcctctctatacttattatagaagaaagttcaaaaatacaagaaactaaaatatttcaattttaatattagctttcgactttgaggataagggatatgtgtacctatgtactcgggttctttcaaagggctttagagaattctcatttgtgcctcttctgtgtggatttagataattttatctgtatgtgactcccctgctgaatgttttatcatattacacatctatgttgaataataaggtctatcaagccgggtggtggtggcacacgcctttaatcccagcacttgggaggcagaggcaggcgaatttctgagttcgaggccagcctggtctacaaagtgagttccaggacagccagggctatacagagaaaccctgtcttgaaaaaaatcaaaataaataaataagtaaataaataaagtctaacaataatagattccctacataattatattccgttacgatcatcatatatggctagtcactgactctcctatatgccagatattctgctgggctctttgaacgtgttagctcatttgctactatctaaggaggtgctgggttctttttaatgtgtgtgatcatgggaagtgtgtattcaccccgaagggctttcacactcggcaaacacagaagccctgatgcttggccgagagatgagaaccagcactcttgggccaaagagatcctgcatgtggagagctttcggggcagtaaggttgggggtggtcgtggtgaccagcaagcaggagaagagcgctaagcagaatgacatggagatctggaactcgcttgagtgccgaggtctgtctagcaagatattaggacctcagagcactgctggcattcacctgatggagtgcatctggcaacaagtagtgatgcttggctaatggtcattgtcctctcaaagaactttattgtcagcagaagtttttcaagttatttctcttcgatggtgccttacaaaagaaagatctgccctttgccctcggaggctcagactttgttaagcaggcattatctgtattgtttctctctgatctttggtgagatgtgtttctcattctcgtaggtaaggctagcatgtggtgtaacgaagcaaacaaacccagctctaccacgcgtgttagcgtgtgactgtgaaggctgcttagtgccactggccttccatcctcatgtacatcaaaaataaaataaaataaaataaaataaaataaaataaaacaaaaccaaataaaaggtgcccagcagagctaacctggttactgggtgaacttgcatttgtacttcagatgctcttcaggttttcccagttgggagcgcagagcactccggtggaaggccatccatcctgctagccacagaccctaacctcccgtttcttttcctccttcctccctttagtgatcgatctagaaactggaaaaacaagtgcttctcgaccacagacaccgagtgcgacctcacagacgagatcgtgaaggatgtgacctgggcctatgaagcaaaggtcctctctgtcccacggaggaactcagttcatggagacggagaccaacttgtgattcatggggaggagccgccatttacaaacgccccaaagtttttaccttaccgagacagtaagtagcttggtttgtaatgccttcactcttggtctaggcttctgtataaatatgcttgtgcttcatgaccccctattagccattaattagatctctctctctctctctctctctctctctctctctctctctctctctcacacacacacacacacacacacacacacacacacacacacaccaaggagatgcatcttataagtgtctctctctctgcagatgggcaggaggttcacagaatgtgaactcgtgtcttgatcactatgtcatctactcactttctcctccagtctgccgccacttcctatcaggaaccagctggagggtgcgctaaagctggggttgtggcttttcctcaggctactgtgtcatatgctgaattttatattctttcatcagaccaggggtgctagtttagacattttcctccaatgagcacagatccacagggaagccacttgtgcctgaaattgggcttgtcagcctccctcgtgctgggtttgaatgtgtagcatgctttcccacctttgtccaagtgtgtgagcctgccatctaacaaggaaatcttctttcctttctctctctctctctctctctctctctctctgtgtgtgtgtgtgtgtgtgtatgtatgtatatcttccttcctttcttttttctttccttccatgtttcagaagtagacatttcacaagcttatgcagcacattaggaagactaaatcccacttcttttgcaaggcctgctggttaaaacacaagtgttgtggttttggtaatccccatctgtccccttctgattgctcagcgacgtggatggtgagaaggatgaagggcaattgagaagattttacttccgatgtttgctctaatggtctaactacccaaatgaatgtagctgccttcctggggtccccactgacaagcctctcttcggactcctttgtatttttacctttgtgcttctgcacaacacgcgcatgccaatcatgctccccaagctgtgatttcccttcatagttcttgagctttcagcagtccagaccaaatcctttgagatacacaacagaacacacacaatcacacacacacacacacactgttctgtgatttgcacagagtgaggacttgtctgaccatcacatgtgctgcagttacctcgtgtaactgaatcaggaaccaagaacttaaaatagattgggtgtgggatgtgtcaagaggttgttgcaggaaaacattaaaaattaacaaaattagaaggtgacaaatgtgcacatagtggactttcaaaaaaaaaagtctatgcctggtgtatgtttgtgtatcatctgtaactcagttccaatgatcagatgccatctgacctgtggaccctagccatgtatgtagtacacacatccatagaacaggcaaaatacatgcataaaaataaaaaaatatttttaaaagtcttaaaaaaaaaaaaaaacccttcctgggcttcccagcctctaatgcattgtagctaaaatacggaagcttggaaaacttcttgtccagcctggtagccatcactcacacatggctaccaaacttttgaaatatggctaatgttgccaaggtgaaaaatatatgccagtttttgaaaaatataaaatatccataatatccactatattgagtgcatgttaaaatgccaatctgttaatggacctactaaagtatataattaaagttaattccacaggctccttctcacatttttctaacatctgcaaggaagggtctccttgtgcttccagaggacggcatggcttgctatgtgttgagactcagctacaagaccgtcaccactcctgctactggagctgattctgtacaggtgttctgtatgagcgatggaggagctgtcaattacgataactaagtgatcttttctttgtattgatttttaatttctaagcaaggtatttttttttccatttcagcaaaccttggacagccagtaattcagcagtttgaacaagatggtagaaaactgaacgtggttgtaaaagactcacttacattagtcagaaagaatggtacattcctcaccctgcggcaagtctttggcaaggacttgggttatataattacttatcggaaaggctcaagcacgggaaaggtaagagattttccatttattttatgccccccccctttttaatagatctttgattctccacagcatgcttgcttcccagttcaaatatggaggaatgggggttgacatggtggtgtgcataacaggtcctccttctgcctgtcactgctgaaccttctgcccttgggcagggtcctttcagagattccagtcacacagtcaaggagctagaccagactgtcctctcccacatcttcatattggggaccccagggtgggggtggagcagggataacttattaactaatttgcatttgtgtgcacatgtgtatgaatgcatgtacaggcatgtgtacatgtgcatgagagtttgtatgtgcatgtgtgtgcgcatgtgcatgtatgtgcttgcgtgtgtgatgctgggcagtgttacccaggaccttgtacactagacaagttctctaccactgagttgcattcccagcccccaaatgccccttctttggtgattccatctctcttttcctacagtaaaaaggggctgaactgggctaggtgtactgtctttttagcagtcatcaggggttaccgatgtgccaagtgacttctaatccctcgtgcgtcaatgatgtctgcatggacttagagccctggttttgcttttctcggtgatttgagaactctatcttttcacagaaaacaaacattacaaacaccaatgaattctcgattgatgtggaagaaggagtaagctactgcttttttgtacaagctatgattttctccaggaaaactaaccaaaatagcccaggaagcagtacagtgtgcaccgagcaatggaagagtttcctgggaggtgagtggccatggctgtcagtgccctgggaggctcggctgcccccacatcaaagccaacatgtatctcttagtttcaaacattcagaaatgagcttagggtgtcttagaatgtctttttggattcttcattctctttgggggcatttaggaatgatttcacattgttcccttggtcacttccctgggcacttgcaatgggccaggttctatagggtactgagcatgcagattacagactttagaacttaggtaccaagtcacagccttctctgcctgtttttcctactgacatgatctgaggggcctgggcccaggggaggagtagtttctggaccagctagtttctggaatggcagatatttcagtgcacctatgatggactgttggtacaggagctgtgtgtgtgtgtgtgtgtgtgtgtgtgtgtgtgtgtgtgtgtgcatgaatacactctctgggtattcagtgttgtgactaaaagctctgggccccacagagaaggggctctgctgtcagaggagacgactaagggggcagtagctatggcctggctctgttagagcttcaagtcattatgccaactaggccagactcgggaagacaaatgccacatcttcctcttatatgtggaaagctagattaaatgtatgtacatgtatgtatttatatatgtatgtagtggggtcatgaaactagaaaggagaccatgggagagaaggaagaaatgtaaaggggaagggttaataaaatagaagtactgaaaagaggcgggggatggaggaactatctagggaaagaaaccagaaagagaccaaggggcatgggggccaggaatgaatgagaacaaagtctaattgcatgttcttcataaaaaataccataattaaacacattactttgtatgctggcctaaaaataacttttttaaatgcaataatatgtttaagtattctgccttcttgcctctgttaacagaaagttctctttccattggagcccattcccactctgggctagccagctccttagccacaagcctcactattcccttccctaagccctacactatctagttctgatgccccaggctctgcctgtttccctgcttcatctcttaacccaatagatcctctggggtactgactaccccagccaactactgtctcttcgtctccaccatcagcactgtgccccctccttctctgtctttcccatttatcttctgttataatctctgccaccaaactcagggtcgttttttggactacatttccaacatgtgttcttccatccatctcccgtggctcctttctagtccaggaaccttgggtagctcatttaatgctgtagccactgtcagtttctctcaagcgtatgaggtcagataaattcttaatcaaagaatgagagaaagagtggaagaaataaatgtcgggtttcaaaaaggcgggacggtatgatttggggtcacgcccaccggcaagtggcgttcgagcctccatgttgactttacatctttgatttatttttttgtttgtttgtttgtctgtttttgcttttcagaaacactcatcattgtgggagcagtggtgctcctggccaccatctttatcatcctcctgtccatatctctgtgcaagcgcagaaagaaccgagcgggacagaaagggaagaacaccccgtcgcgcttggcatagaggaaaggctgaagccgctaacgctcacactgcctgcacggcactgttgcggagagctctgatgggaactgtgcaacatggagcgtggagcctgccgatcctagctcagagaggctgccttcatggcctgttactccagctaacgctttgattccaacactagcatttgtcacgttaggacgaactgaaacggtacaaactggttaacactacagcgccttttgcacaaatgctttagattgtatggtctacactcaggaagacactaggtcacccaggcaaagccagtggacagatgcctttcatataacctgggtgggcttttggaaaatctttgagaagttgatttcataggctgtagaacagtaaagtgggaactgggcggacttttctaacagtcgtacttttataaagcggtatttgggtgttttttccctcgaataggtacttttggaagttcaaagcaagtggcaaactttcatataaacatgttaaatgcaggatatttctgcttggggcatctttgtgatttgtactttcctacaatttagcactttaactgacaatgatggggtttaaacatttgacagccaactctatttttatacgactactatacaaagaaactacatatagttttatgatttaaggtacttagaaatgtttatggttaacattgtatatatttacataaaatttaaaggttttgtatatgggattttctatttatatagcttctatttgtatattttgagataatttatttaatatacttttatataaataaaggtgattgggaattgagacaatcgcatttatatcttccacttattatttacttcttggttttcacattaggtgactccgttgctgtaacaattgaccttatcagtcttagtggcttacaacaatatatacttttcgcttagactacgtgtttgtgagtgttggtaggtggtagctcctaatggtgtcttctcattacaaggcccgaaacaaagcaggggtctctattggatcagagcaaaggtgaacacacacacacacacatgcacgcgcgcacacacacacacacacacacacacacacacacacacatacatgcgtacacccccccccccccaaagcacaaccataccaaggcaaacaaaacaaaacagcaaaacaggctgctacaccttcctttttttttagtttttcgagacagggtttctctctgtatagccctggctgtcctagaactctctctgtagaccaggctggcctcgaactcagaagtccgcctgcctctgcctctccagtgctgggattaaaggcgtgtgccaccacgcccggctacaccttccatgtttaagccactttttaaaaatgactttttgattataattacatcacttccccccttccctttcctccctgcagtccctccctcgtaatccctgccctttgacccttagactcattcgaattcatagtcttaacacttccaccgaatactttggttcaccagtcaagcaagtcacgtgggtaagcctgataaacctagcctactgagagagaccatctagtacagacttagggttctccccagtcactcacttgcattttggaggaggctcaaagaaaaagagacctgccttagattttgttgttgttgttgataaaaattggaaggatcaagttagacctcatgtgaggaaactaagcacaaggactcggtcatgctgagttgactcagtgacttctaaaacggagaaacggagcgagtccttcagaggcagcaaaagtctacactgcgcagaaagaagatctgaactgaatgatggtgttctgagctggctgccaggcaaacctgctcttgcttccctccctctgtgcaaggctggggagttgccagtggccctcttaactggtcggttttgtttactcaataacaaatgcgctccatctgccccgctgcgggcaccagtgcagcttttgataccacagcgaccagtgtgtgtggtggagtcccaaagagaaaggacaagcatgtgtccttgtcctcagagaggggccttacgccttatcttgagtccctgctcacatgacttatccaggacaaagagctcggtgtagcacacgaagggcaaagttggctacaggagctcatgactgaccttgccttcctctgccaggaataattatagcattatgtaatcccctagcttcataatctaaggaatttgaattatctgcagactgaaagcaggtctggatgtaacaaggtagagcaggaggaagtcgttatagatgcggtgtagcggtgctgcgggaatcagaaggttccactcccagcgttaaccattgcacacaggccccctctctccttgttgtggacatgagacagctgctcctaaatcaacacaacgggttctgcagccgaactgctgccctctagtgggccgaactgaacgtctccctccctgtgctggagcctatcaagcatactatattaattactaaacagaactcgaaagctctctacagatacattttccatcttaaattactgtttggcatatatgtaagagactctttttgaactctgagtcagagcaactctctcttaaacaatcatcttgctcccttagggaaaactcatttctgaatgcccttcctatgagctcctggatttaacacgtactctttaacacattctcttcccccaactgttgcctcgctgtttctttttttctttattataatcttggctttaatagagtgggatcaatcctatttaaggagaggctcccgcaaatgaccagtatgcacatttgtttaacatagcaggagccctcacttaatcacacacaaaaggatgctctgactgggtgctgcacacagatgatctgtagagcaaaatgagaagacaagttggggacgtgacagactcctggaatacacttcacccaaagcccccaccactggaaacccaactaattattttagcaagctggagctgggttttctgttccctgtgatcaagccttctgtcctttgtagttaaaaagaaatacccaaactaacattgtattatgaaagtcaagcataaagaaagaagccagctatggtggtttgaatgagaacggcccccataggctcatatgttcaagtgcatggttcccagctggtggcattatttgggaaagattggccatggtgggtgtggccttgttggagtttgttgctgctggtggactttgaggctgaaaagtccattccattcctctctctctctctctctctctctctctctctctctctctctctctctctctccccccatcccaaccgtcaaagataccaagaactaaaccaccaacccaagagtacacacgcagggcccca